(19) United States
(12) Patent Application Publication
(10) Pub. No.: US 2006/0270670 A1
(43) Pub. Date: Nov. 30, 2006

(54) Methods of synthesizing 6-alkylaminoquinoline derivatives

(76) Inventors: Warren Chew, Outremont (CA); Youchu Wang, St. Laurent (CA); Sridhar Krishna Rabindran, Chestnut Ridge, NY (US); Carolyn Discafani-Marro, Cortlandt Manor, NY (US); John Patrick McGinnis II, New Windsor, NY (US); Allan Wissner, Ardsley, NY (US)

Correspondence Address: FITZPATRICK CELLA (WYETH), 30 ROCKEFELLER PLAZA, NEW YORK, NY, 10112-3800, US

(21) Appl. No.: 11/442,562

(22) Filed: May 25, 2006

Related U.S. Application Data
(60) Provisional application No. 60/685,040, filed on May 25, 2005.

(51) Int. Cl. A61K 31/5377; A61K 31/496; A61K 31/506; A61K 31/4709; C07D 417/02; C07D 403/02; C07D 413/02; C07D 215/38

(52) U.S. Cl. 514/232.5; 514/253.06; 514/256; 514/312; 514/313; 544/126; 544/363; 544/333; 546/153; 546/159

(57) ABSTRACT

The present invention is directed to a method of synthizing compounds of formula (I): 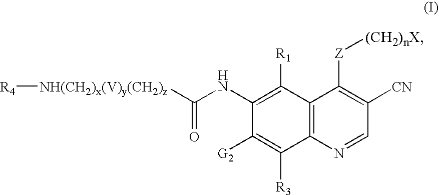
wherein X, Z, V, R₁, R₃, R₄, G₂, n, x, y, and z are defined herein. This invention also includes a method of preparing acid compounds of formula (VII): 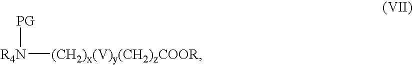
wherein R is H, and R₄, x, y, and z are as defined herein and PG is a protecting group. This invention is also directed to (E)N-{4-[3-chloro-4-(2-pyridinylmethoxy)anilino]-3-cyano-7-ethoxy-6-quinolinyl}-4-(methylamino)-2-butenamide, compositions containing it and methods of using the to treat cancer.

This application claims the benefit of U.S. provisional patent application No. 60/685,040, filed on May 25, 2005.

### BACKGROUND OF THE INVENTION

1. Field of the Invention

This invention is directed to a method of synthesizing a series of substituted quinolines, which are metabolites of EGFR and HER2 kinase inhibitors, and have use in the treatment of cancerous tumors.

2. Related Background Art

Protein kinases are a class of enzymes that catalyze the transfer of a phosphate group from ATP to a tyrosine, serine, threonine, or histidine residue located on a protein substrate, many of which play a role in normal cell growth. Correspondingly, several growth factor receptor proteins function as protein tyrosine kinases (PTKs) to effect signaling and are known as receptor tyrosine kinases (RTKs).

The RTKs comprise one of the larger families of PTKs and have diverse biological activity. At present, at least nineteen (19) distinct subfamilies of RTKs have been identified. One such subfamily is the “HER” family of RTKs, which includes EGFR (epithelial growth factor receptor), HER2, HER3 and HER4. It has been shown that under certain conditions, as a result of either mutation or over expression, these RTKs can become deregulated; the result of which is uncontrolled cell proliferation which can lead to tumor growth and cancer [Wilks, A. F., Adv. Cancer Res., 60, 43 (1993) and Parsons, J. T.; Parsons, S. J., Important Advances in Oncology, DeVita, V. T. Ed., J. B. Lippincott Co., Phila., 3 (1993)]. For example, over expression of the receptor kinase product of the erbB-2 oncogene has been associated with human breast and ovarian cancers [Slamon, D. J. et al., Science, 244, 707 (1989) and Science, 235, 177 (1987)]. In addition, deregulation of EGF-R kinase has been associated with epidermoid tumors [Reiss, M., et al., Cancer Res., 51, 6254 (1991)], breast tumors [Macias, A. et al., Anticancer Res., 7, 459 (1987)], and tumors involving other major organs [Gullick, W. J., Brit. Med. Bull., 47, 87 (1991)]. RTK inhibitors, therefore have potential therapeutic value for the treatment of cancer and other diseases characterized by uncontrolled or abnormal cell growth. Accordingly, many recent studies have dealt with the development of specific RTK inhibitors as potential anti-cancer therapeutic agents [some recent reviews: Traxler, P., Exp. Opin. Ther. Patents, 8, 1599 (1998) and Bridges, A. J., Emerging Drugs, 3, 279 (1998)].

U.S. Pat. Nos. 6,002,008, 6,288,082, and 6,297,258, all of Wissner et al., describe such PTK, and particularly, RTK inhibitor compounds. The compounds of the Wissner et al. patents are all substituted 3-cyanoquinolines. The ‘des-alkyl’ compounds synthesized by the present invention are metabolites of the EGFR kinase inhibitors and the HER2 kinase inhibitors disclosed in Wissner et al.

### BRIEF SUMMARY OF THE INVENTION

The present invention is directed to a method of synthesizing compounds of formula (I): 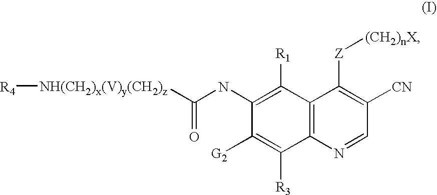
comprising the step of deprotecting a compound of formula (II): 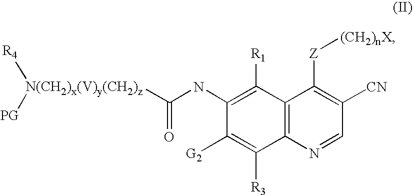
wherein X is cycloalkyl of 3 to 7 carbon atoms, which may be optionally substituted with one or more alkyl of 1 to 6 carbon atom groups; or is a pyridinyl, pyrimidinyl, or phenyl ring; wherein the pyridinyl, pyrimidinyl, or phenyl ring may be optionally mono- di-, or tri-substituted with a substituent selected from the group consisting of halogen, alkyl of 1-6 carbon atoms, alkenyl of 2-6 carbon atoms, alkynyl of 2-6 carbon atoms, azido, hydroxyalkyl of 1-6 carbon atoms, halomethyl, alkoxymethyl of 2-7 carbon atoms, alkanoyloxymethyl of 2-7 carbon atoms, alkoxy of 1-6 carbon atoms, alkylthio of 1-6 carbon atoms, hydroxy, trifluoromethyl, cyano, nitro, carboxy, carboalkoxy of 2-7 carbon atoms, carboalkyl of 2-7 carbon atoms, phenoxy, phenyl, thiophenoxy, benzoyl, benzyl, amino, alkylamino of 1-6 carbon atoms, dialkylamino of 2 to 12 carbon atoms, phenylamino, benzylamino, alkanoylamino of 1-6 carbon atoms, alkenoylamino of 3-8 carbon atoms, alkynoylamino of 3-8 carbon atoms, carboxyalkyl of 2-7 carbon atoms, carboalkoxyalkyl of 3-8 carbon atoms, aminoalkyl of 1-5 carbon atoms, N-alkylaminoalkyl of 2-9 carbon atoms, N,N-dialkylaminoalkyl of 3-10 carbon atoms, N-alkylaminoalkoxy of 2-9 carbon atoms, N,N-dialkylaminoalkoxy of 3-10 carbon atoms, mercapto, and benzoylamino; or X is a bicyclic aryl or bicyclic heteroaryl ring system of 8 to 12 atoms where the bicyclic heteroaryl ring contains 1 to 4 heteroatoms selected from N, O, and S with the proviso that the bicyclic heteroaryl ring does not contain O—O, S—S, or S—O bonds and where the bicyclic aryl or bicyclic heteroaryl ring may be optionally mono- di-, tri, or tetra-substituted with a substituent selected from the group consisting of halogen, oxo, thio, alkyl of 1-6 carbon atoms, alkenyl of 2-6 carbon atoms, alkynyl of 2-6 carbon atoms, azido, hydroxyalkyl of 1-6 carbon atoms, halomethyl, alkoxymethyl of 2-7 carbon atoms, alkanoyloxymethyl of 2-7 carbon atoms, alkoxy of 1-6 carbon atoms, alkylthio of 1-6 carbon atoms, hydroxy, trifluoromethyl, cyano, nitro, carboxy, carboalkoxy of 2-7 carbon atoms, carboalkyl of 2-7 carbon atoms, phenoxy, phenyl, thiophenoxy, benzoyl, benzyl, amino, alkylamino of 1-6 carbon atoms, dialkyl amino of 2 to 12 carbon atoms, phenylamino, benzylamino, alkanoylamino of 1-6 carbon atoms, alkenoylamino of 3-8 carbon atoms, alkynoylamino of 3-8 carbon atoms, carboxyalkyl of 2-7 carbon atoms, carboalkoxyalkyl of 3-8 carbon atoms, aminoalkyl of 1-5 carbon atoms, N-alkylaminoalkyl of 2-9 carbon atoms, N,N-dialkylaminoalkyl of 3-10 carbon atoms, N-alkylaminoalkoxy of 2-9 carbon atoms, N,N-dialkylaminoalkoxy of 3-10 carbon atoms, mercapto, and benzoylamino; or X is a radical having the formula: 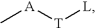
wherein A is a pyridinyl, pyrimidinyl, or phenyl ring; wherein the pyridinyl, pyrimidinyl, or phenyl ring may be optionally mono- or di-substituted with a substituent selected from the group consisting of halogen, alkyl of 1-6 carbon atoms, alkenyl of 2-6 carbon atoms, alkynyl of 2-6 carbon atoms, azido, hydroxyalkyl of 1-6 carbon atoms, halomethyl, alkoxymethyl of 2-7 carbon atoms, alkanoyloxymethyl of 2-7 carbon atoms, alkoxy of 1-6 carbon atoms, alkylthio of 1-6 carbon atoms, hydroxy, trifluoromethyl, cyano, nitro, carboxy, carboalkoxy of 2-7 carbon atoms, carboalkyl of 2-7 carbon atoms, phenoxy, phenyl, thiophenoxy, benzoyl, benzyl, amino, alkylamino of 1-6 carbon atoms, dialkylamino of 2 to 12 carbon atoms, phenylamino, benzylamino, alkanoylamino of 1-6 carbon atoms, alkenoylamino of 3-8 carbon atoms, alkynoylamino of 3-8 carbon atoms, carboxyalkyl of 2-7 carbon atoms, carboalkoxyalkyl of 3-8 carbon atoms, aminoalkyl of 1-5 carbon atoms, N-alkylaminoalkyl of 2-9 carbon atoms, N,N-dialkylaminoalkyl of 3-10 carbon atoms, N-alkylaminoalkoxy of 2-9 carbon atoms, N,N-dialkylaminoalkoxy of 3-10 carbon atoms, mercapto, and benzoylamino; T is bonded to a carbon of A and is:

—NH(CH₂)ₘ—, —O(CH₂)ₘ—, —S(CH₂)ₘ—, —NR(CH₂)ₘ—, —(CH₂)ₘ—, —(CH₂)ₘNH—, —(CH₂)ₘO—, —(CH₂)ₘS—, or —(CH₂)ₘNR—;
L is an unsubsitituted phenyl ring or a phenyl ring mono-, di-, or tri-substituted with a substituent selected from the group consisting of halogen, alkyl of 1-6 carbon atoms, alkenyl of 2-6 carbon atoms, alkynyl of 2-6 carbon atoms, azido, hydroxyalkyl of 1-6 carbon atoms, halomethyl, alkoxymethyl of 2-7 carbon atoms, alkanoyloxymethyl of 2-7 carbon atoms, alkoxy of 1-6 carbon atoms, alkylthio of 1-6 carbon atoms, hydroxy, trifluoromethyl, cyano, nitro, carboxy, carboalkoxy of 2-7 carbon atoms, carboalkyl of 2-7 carbon atoms, phenoxy, phenyl, thiophenoxy, benzoyl, benzyl, amino, alkylamino of 1-6 carbon atoms, dialkylamino of 2 to 12 carbon atoms, phenylamino, benzylamino, alkanoylamino of 1-6 carbon atoms, alkenoylamino of 3-8 carbon atoms, alkynoylamino of 3-8 carbon atoms, carboxyalkyl of 2-7 carbon atoms, carboalkoxyalkyl of 3-8 carbon atoms, aminoalkyl of 1-5 carbon atoms, N-alkylaminoalkyl of 2-9 carbon atoms, N,N-dialkylaminoalkyl of 3-10 carbon atoms, N-alkylaminoalkoxy of 2-9 carbon atoms, N,N-dialkylaminoalkoxy of 3-10 carbon atoms, mercapto, and benzoylamino; or L is a 5- or 6-membered heteroaryl ring where the heteroaryl ring contains 1 to 3 heteroatoms selected from N, O, and S, with the proviso that the heteroaryl ring does not contain O—O, S—S, or S—O bonds, and where the heteroaryl ring is optionally mono- or di-substituted with a substituent selected from the group consisting of halogen, oxo, thio, alkyl of 1-6 carbon atoms, alkenyl of 2-6 carbon atoms, alkynyl of 2-6 carbon atoms, azido, hydroxyalkyl of 1-6 carbon atoms, halomethyl, alkoxymethyl of 2-7 carbon atoms, alkanoyloxymethyl of 2-7 carbon atoms, alkoxy of 1-6 carbon atoms, alkylthio of 1-6 carbon atoms, hydroxy, trifluoromethyl, cyano, nitro, carboxy, carboalkoxy of 2-7 carbon atoms, carboalkyl of 2-7 carbon atoms, phenoxy, phenyl, thiophenoxy, benzoyl, benzyl, amino, alkylamino of 1-6 carbon atoms, dialkylamino of 2 to 12 carbon atoms, phenylamino, benzylamino, alkanoylamino of 1-6 carbon atoms, alkenoylamino of 3-8 carbon atoms, alkynoylamino of 3-8 carbon atoms, carboxyalkyl of 2-7 carbon atoms, carboalkoxyalkyl of 3-8 carbon atoms, aminoalkyl of 1-5 carbon atoms, N-alkylaminoalkyl of 2-9 carbon atoms, N,N-dialkylaminoalkyl of 3-10 carbon atoms, N-alkylaminoalkoxy of 2-9 carbon atoms, N,N-dialkylaminoalkoxy of 3-10 carbon atoms, mercapto, and benzoylamino; V is ethylene or acetylene; PG is an amine protecting group; R₄ is alkyl of 1 to 6 carbons; Z is NR^{z′}, O or S, wherein R^{z′} is H or C₁-C₆ alkyl; R₁, G₂, and R₃ are each, independently, hydrogen, halogen, alkyl of 1-6 carbon atoms, alkenyl of 2-6 carbon atoms, alkynyl of 2-6 carbon atoms, alkenyloxy of 2-6 carbon atoms, alkynyloxy of 2-6 carbon atoms, hydroxymethyl, halomethyl, alkanoyloxy of 1-6 carbon atoms, alkenoyloxy of 3-8 carbon atoms, alkynoyloxy of 3-8 carbon atoms, alkanoyloxymethyl of 2-7 carbon atoms, alkenoyloxymethyl of 4-9 carbon atoms, alkynoyloxymethyl of 4-9 carbon atoms, alkoxymethyl of 2-7 carbon atoms, alkoxy of 1-6 carbon atoms, alkylthio of 1-6 carbon atoms, alkylsulphinyl of 1-6 carbon atoms, alkylsulphonyl of 1-6 carbon atoms, alkylsulfonamido of 1-6 carbon atoms, alkenylsulfonamido of 2-6 carbon atoms, alkynylsulfonamido of 2-6 carbon atoms, hydroxy, trifluoromethyl, trifluoromethoxy, cyano, nitro, carboxy, carboalkoxy of 2-7 carbon atoms, carboalkyl of 2-7 carbon atoms, phenoxy, phthalimide, phenyl, thiophenoxy, benzyl, amino, hydroxyamino, alkoxyamino of 14 carbon atoms, alkylamino of 1-6 carbon atoms, dialkylamino of 2 to 12 carbon atoms, N-alkylcarbamoyl, N,N-dialkylcarbamoyl, N-alkyl-N-alkenylamino of 4 to 12 carbon atoms, N,N-dialkenylamino of 6-12 carbon atoms, phenylamino, benzylamino, 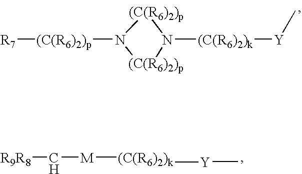
R₇—(C(R₆)₂)_{g}—Y—, R₇—(C(R₆)₂)ₚ-M-(C(R₆)₂)ₖ—Y—, or Het-(C(R₆)₂)_{q}W—(C(R₆)₂—Y—; or R₁ and R₃ are as defined above and G₂ is R₂—NH—; or if any of the substituents R₁, G₂ or R₃ are located on contiguous carbon atoms then they may be taken together as the divalent radical —O—C(R₆)₂—O—; Y is a divalent radical selected from the group consisting of 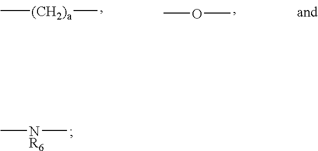
R₇ is —NR₆R₆, —OR₆, -J, —N(R₆)3+, or —NR₆(OR₆); M is >NR₆, —O—, >N—(C(R₆)₂)ₚNR₆R₆, or >N—(C(R₆)₂)ₚ—OR₆; W is >NR₆, —O— or is a bond; Het is is selected from the group consisting of morpholine, thiomorpholine, thiomorpholine S-oxide, thiomorpholine S,S-dioxide, piperidine, pyrrolidine, aziridine, pyridine, imidazole, 1,2,3-triazole, 1,2,4-triazole, thiazole, thiazolidine, tetrazole, piperazine, furan, thiophene, tetrahydrothiophene, tetrahydrofuran, dioxane, 1,3-dioxolane, tetrahydropyran, and 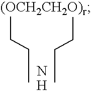
wherein Het is optionally mono- or di-substituted on carbon or nitrogen with R₆, optionally mono- or di-substituted on carbon with hydroxy, —N(R₆)₂, or —OR₆, optionally mono or di-substituted on carbon with the mono-valent radicals —C(R₆)₂)ₛOR₆ or —(C(R₆)₂)ₛ N(R₆)₂, and optionally mono or di-substituted on a saturated carbon with divalent radicals —O— or —O(C(R₆)₂)ₛ—O—; R₆ is hydrogen, alkyl of 1-6 carbon atoms, alkenyl of 2-6 carbon atoms, alkynyl of 2-6 carbon atoms, cycloalkyl of 1-6 carbon atoms, carboalkyl of 2-7 carbon atoms, carboxyalkyl (2-7 carbon atoms), phenyl, or phenyl optionally substituted with one or more halogen, alkoxy of 1-6 carbon atoms, trifluoromethyl, amino, alkylamino of 1-3 carbon atoms, dialkylamino of 2-6 carbon atoms, nitro, cyano, azido, halomethyl, alkoxymethyl of 2-7 carbon atoms, alkanoyloxymethyl of 2-7 carbon atoms, alkylthio of 1-6 carbon atoms, hydroxy, carboxyl, carboalkoxy of 2-7 carbon atoms, phenoxy, phenyl, thiophenoxy, benzoyl, benzyl, phenylamino, benzylamino, alkanoylamino of 1-6 carbon atoms, or alkyl of 1-6 carbon atoms; with the proviso that the alkenyl or alkynyl moiety is bound to a nitrogen or oxygen atom through a saturated carbon atom; R₂, is selected from the group consisting of 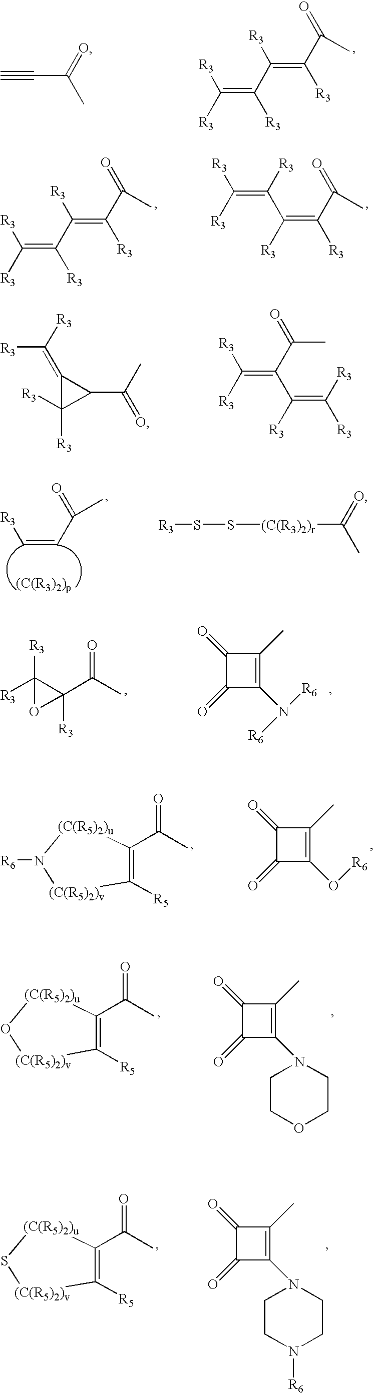 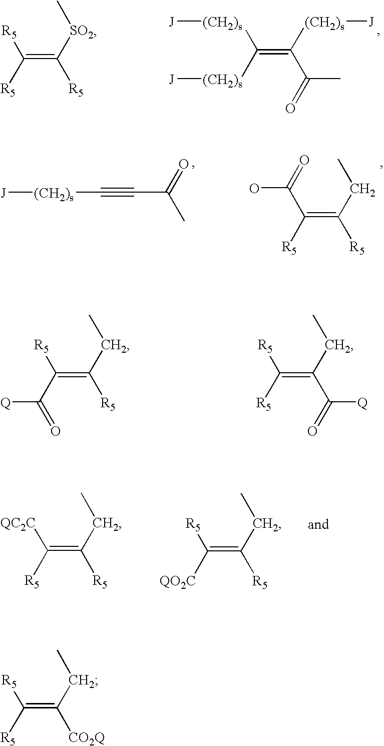
R₃ is independently hydrogen, alkyl of 1-6 carbon atoms, carboxy, carboalkoxy of 1-6 carbon atoms, phenyl, carboalkyl of 2-7 carbon atoms, 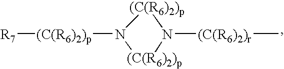
R₇—(C(R₆)₂)ₛ—, R₇—(C(R₆)₂)ₚ-M-(C(R₆)₂)ᵣ—, R₈R₉—CH—, M-(C(R₆)₂)ᵣ—, or Het-(C(R₆)₂)_{q}—W—C(R₆)₂)ᵣ—; R₅ is independently hydrogen, alkyl of 1-6 carbon atoms, carboxy, carboalkoxy of 1-6 carbon atoms, phenyl carboalkyl of 2-7 carbon atoms, 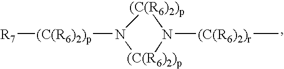
R₇—(C(R₆)₂)ₛ—, R₇—(C(R₆)₂)ₚ-M-(C(R₆)₂)ᵣ—, R₈R₉ —CH—, M-(C(R₆)₂)ᵣ—, or Het-(C(R₆)₂)_{q}—W—(C(R₆)₂)ᵣ—; R₈, and R₉ are each independently —(C(R₆)₂)ᵣNR₆R₆, or —(C(R₆)₂)ᵣOR₆; J is independently hydrogen, chlorine, fluorine, or bromine; Q is aklyl of 1-6 carbon atoms or hydrogen; a=0 or 1; g=1-6; k=0-4; n is 0-1; m is 0-3; p=2-4; q=0-4; r=1-4; s=1-6; u=0-4 and v=0-4, wherein the sum of u+v is 2-4; x=0-3; y=0-1; and z=0-3; or a pharmaceutically acceptable salt thereof.

The present invention is also directed to a method of preparing a compound of formula (I): 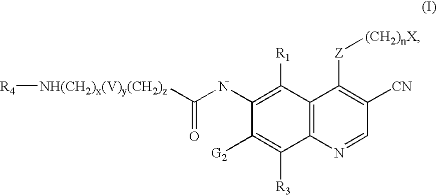
comprising the step of coupling an anilinoquinoline of formula (III): 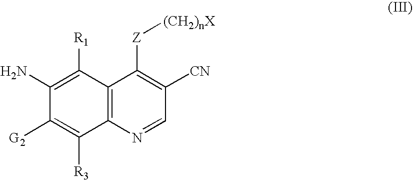
with an acid of formula (VII″):
R₄—NH.(CH₂)ₓ(V)_{y}(CH₂)_{z}COOR″ (VII″)
wherein R″ is H, and R₁, R₃, R₄, Z, G₂, V, n, x, y, and z are as previously defined.

This invention also pertains to a method of preparing acid compounds of formula (VII): 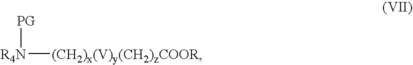
wherein R is H, and R₄, V, x, y, and z are as defined above and PG is an amine protecting group, comprising the step of hydrolysizing a corresponding ester of formula (VII′): 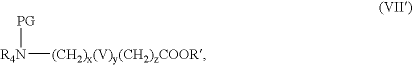
wherein R′ is alkyl of 1 to 6 carbon atoms or aryl to form the acid of formula (VII).

This invention is also directed to (E)N-{4-[3-chloro-4-(2-pyridinylmethoxy)anilino]-3-cyano-7-ethoxy-6-quinolinyl}-4-(methylamino)-2-butenamide, or a pharmaceutically acceptable salt thereof. Also contemplated by the present invention are compositions containing the same and methods of using this compound to prevent, treat or inhibit cancers.

### DETAILED DESCRIPTION OF THE INVENTION

The 6-des-alkylaminoquinolines of formula (I) are metabolites of EGFR and HER2 inhibitors, and possess EGFR and HER2 inhibitory activity themselves. A particularly preferred embodiment of the present invention is directed to a method of making these compounds by first arylating a protected 6-anilino-3-cyanoquinoline at the 4-position with a reagent of formula HZ-(CH₂)ₙX, which is defined herein. Then the aniline nitrogen is deprotected and the compound is coupled to an acid of formula (R₄)(PG)N—(CH₂)ₓ(V)_{y}(CH₂)_{z}COOH using standard coupling reagents to form the 6-amidoquinoline of formula (II). The protected secondary amine of this compound can then be deprotected to give the desired product. Alternatively, the 6-des-alkylaminoquinolines of formula (I) can be formed directly by coupling the aniline of formula (III) with unprotected acid of formula (VII″), R₄—NH—(CH₂)ₓ(V)_{y}(CH₂)_{z}COOH, using standard coupling reagents.

This method is an improvement over the prior method, which alkylated the 6-anilinoquinoline compound using an acid chloride. The prior method produced poor yields because of the instability of the acid compounds under the reaction conditions. In addition, the prior method also produced a crude product that was exetremely difficult to purify. The present method, however, gives reasonable yields and a crude product that can easily be made >97% pure with one flash chromatography.

For purposes of this invention the term “alkyl”, unless stated otherwise, includes both straight and branched alkyl moieties, which can contain as many as 12 carbon atoms. Preferably, the alkyl moiety contains between 1 to 6 carbon atoms, though 1 to 4 carbon atoms is more preferable. The term “alkenyl” refers to a radical aliphatic hydrocarbon containing one double bond and includes both straight and branched alkenyl moieties of 2 to 7 carbon atoms. Such alkenyl moieties may exist in the E or Z configurations; the compounds of this invention include both configurations. The term “alkynyl” includes both straight chain and branched moieties containing 2 to 6 carbon atoms having at least one triple bond. The term “cycloalkyl” refers to alicyclic hydrocarbon groups having 3 to 12 carbon atoms, but is preferably 3 to 7 carbon atoms, and includes but is not limited to: cyclopropyl, cyclobutyl, cyclopentyl, cyclohexyl, cycloheptyl, norbornyl, or adamantyl.

For purposes of this invention the term “aryl” is defined as an aromatic hydrocarbon moiety, which may be a single ring or a multiple fused ring system in which all the double bonds are in conjugation, and may be substituted or unsubstituted. An aryl group preferably contains 6 to 12 carbon atoms and may be selected from, but not limited to, the group: phenyl, α-naphthyl, β-naphthyl, biphenyl, anthryl, tetrahydronaphthyl, phenanthryl, fluorenyl, indanyl, biphenylenyl, acenaphthenyl, acenaphthylenyl, or phenanthrenyl groups. An aryl group may be optionally mono-, di-, tri- or tetra-substituted with substituents selected from, but not limited to, the group consisting of alkyl, acyl, alkoxycarbonyl, alkoxy, alkoxyalkyl, alkoxyalkoxy, cyano, halogen, hydroxy, nitro, trifluoromethyl, trifluoromethoxy, trifluoropropyl, amino, alkylamino, dialkylamino, dialkylaminoalkyl, hydroxyalkyl, alkoxyalkyl, alkylthio, —SO₃H, —SO₂NH₂, —SO₂NHalkyl, —SO₂N(alkyl)₂, —CO₂H, CO₂NH₂, CO₂NHalkyl, and —CO₂N(alkyl)₂. Preferred substituents for aryl include: alkyl, halogen, amino, alkylamino, dialkylamino, trifluoromethyl, trifluoromethoxy, arylalkyl, and alkylaryl.

For purposes of this invention the term “heteroaryl” is defined as an aromatic heterocyclic ring system (monocyclic or bicyclic) where the heteroaryl moieties are five or six membered rings containing 1 to 4 heteroatoms selected from the group consisting of S, N, and O, and include but is not limited to: (1) furan, thiophene, indole, azaindole, oxazole, thiazole, isoxazole, isothiazole, imidazole, N-methylimidazole, pyridine, pyrimidine, pyrazine, pyrrole, N-methylpyrrole, pyrazole, N-methylpyrazole, 1,3,4-oxadiazole, 1,2,4-triazole, 1-methyl-1,2,4-triazole, 1H-tetrazole, 1-methyltetrazole, benzoxazole, benzothiazole, benzofuran, benzisoxazole, benzimidazole, N-methylbenzimidazole, azabenzimidazole, indazole, quinazoline, quinoline; (2) a bicyclic aromatic heterocycle where a phenyl, pyridine, pyrimidine or pyridizine ring is: (i) fused to a 6-membered aromatic (unsaturated) heterocyclic ring having at least one heteroatom; (ii) fused to a 5-membered aromatic or nonaromatic (unsaturated) heterocyclic ring having at least one heteroatom selected from O, N or S. Preferably a bicyclic heteroaryl group contains 8 to 12 carbon atoms. Preferred substituents for heteroaryl include: alkyl, halogen, amino, alkylamino, dialkylamino, trifluoromethyl, trifluoromethoxy, arylalkyl, and alkylaryl.

For the purposes of this invention the term “heterocycloalkyl” refers to a non-aromatic heterocyclic ring system (monocyclic or bicyclic) where the moieties contain 1 to 4 heteroatoms selected from the group consisting of S, N, and O, and include but is not limited to: Pyrrolidine, pyrroline, 1,3-dioxolane, imidazoline, imidazolidine, pyrazoline, pyrazolidine, pyran, piperidine, dioxane, morpholine, dithioxane, thiomorpholine, piperazine, azetidinyl, hexahydroazepinyl, dihydrobenzimidazolyl, dihydrobenzofuranyl, dihydrobenzothienyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, dihydro-1,4-dioxanyl, tetrahydrofuranyl, tetrahydrothienyl, tetrahydroquinolinyl, tetrahydroisoquinolinyl and indoline. A heterocycloalkyl moiety preferably contains 1-11 carbon atoms. These moieties may be further substituted.

For the purposes of this invention the term “alkoxy” is defined as C₁-C₆-alkyl-O—; the term “aryloxy” is defined as aryl-O—; the term “heteroaryloxy” is defined as heteroaryl-O—; wherein alkyl, aryl, and heteroaryl are as defined above.

For purposes of this invention the term “arylalkyl” is defined as aryl-C₁-C₆-alkyl-; arylalkyl moieties include benzyl, 1-phenylethyl, 2-phenylethyl, 3-phenylpropyl, 2-phenylpropyl and the like.

For purposes of this invention the term “alkanoyloxymethyl” is defined as —CH₂OC(O)R, wherein R is alkyl of 1 to 6 carbon atoms.

For purposes of this invention the term “alkylthio” is defined as C₁-C₆-alkyl-S.

For purposes of this invention “alkylthioalkyl,” and “alkoxyalkyl,” denote an alkyl group as defined above that is further substituted with an alkoxy or alkylthio as defined above.

The terms “alkylamino” and “dialkylamino” refer to moieties with one or two alkyl groups wherein the alkyl chain is 1 to 6 carbons and the groups may be the same or different. The terms “monoalkylaminoalkyl” and “dialkylaminoalkyl” refer to monoalkylamino and dialkylamino moieties with one or two alkyl groups (the same or different) bonded to the nitrogen atom which is attached to an alkyl group of 1 to 6 carbon atoms. Preferably a dialkylaminoalkyl moiety consist of 3 to 10 carbon atoms and a alkylaminoalkyl moiety consist of from 2 to 9 carbon atoms.

The terms “alkylaminoalkoxy” and “dialkylaminoalkoxy” refer to alkylamino and dialkylamino moieties with one or two alkyl groups (the same or different) bonded to the nitrogen atom which is attached to an alkoxy group of 1 to 6 carbon atoms. Preferably a dialkylaminoalkoxy moiety consist of 3 to 10 carbon atoms and a alkylaminoalkoxy moiety consist of from 2 to 9 carbon atoms.

For purposes of this invention the term “benzoylamino” is defined as a Ph-OC(O)NH— moiety.

For purposes of this invention the term “carboxy” is defined as a —COOH moiety.

For purposes of this invention the term “alkanoylamino” is defined as a —NH—COOR moiety, wherein R is alkyl of 1 to 6 carbon atoms.

For purposes of this invention the term “alkenoylamino” and “alkynoylamino” are defined as a —NH—COOR moiety, wherein R is alkenyl or alkynyl of 3 to 8 carbon atoms.

For purposes of this invention the term “carboalkoxy” is defined as —CO₂R, wherein R is alkyl of 1 to 6 carbon atoms.

For purposes of this invention the term “carboalkyl” is defined as —COR, wherein R is alkyl of 1 to 6 carbon atoms.

For purposes of this invention the term “carboxyalkyl” is defined as a HOOCR— moiety, wherein R is alkyl of 1 to 6 carbon atoms.

For purposes of this invention the term “carboalkoxyalkyl” is defined as a —R—CO₂—R′ moiety, wherein R and R′ are alkyl and together consist of from 2 to 7 carbon atoms.

For purposes of this invention the term “aminoalkyl” is defined as H₂N-alkyl, wherein the alkyl group consist of 1 to 5 carbon atoms.

“Azido” is a radical of the formula —N₃.

“Acyl” is a radical of the formula —(C═O)-alkyl or —(C═O)-perfluoroalkyl wherein the alkyl radical or perfluoroalkyl radical is 1 to 6 carbon atoms; preferred examples include but are not limited to, acetyl, propionyl, butyryl, trifluoroacetyl.

For purposes of this invention the term “alkylsulfinyl” is defined as a R′SO— radical, where R′ is an alkyl radical of 1 to 6 carbon atoms. Alkylsulfonyl is a R′SO₂— radical, where R′ is an alkyl radical of 1 to 6 carbon atoms. Alkylsulfonamido, alkenylsulfonamido, alkynylsulfonamido are R′SO₂NH— radicals, where R′ is an alkyl radical of 1 to 6 carbon atoms, an alkenyl radical of 2 to 6 carbon atoms, or an alkynyl radical of 2 to 6 carbon atoms, respectively.

Saturated or partially saturated non-aromatic heteroaryl groups are defined in this invention as heterocyclic rings selected from but not limited to the moieties: azetidinyl, 1,4-dioxanyl, hexahydroazepinyl, piperazinyl, piperidinyl, pyrrolidinyl, morpholinyl, thiomorpholinyl, dihydrobenzimidazolyl, dihydrobenzofuranyl, dihydrobenzothienyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, dihydro-1,4-dioxanyl, tetrahydrofuranyl, tetrahydrothienyl, tetrahydroquinolinyl, and tetrahydroisoquinolinyl. Preferably such moieties contain 3-10 ring atoms, wherein 1 to 4 are heteroatoms selected from the group consisting of S, N and O.

The term “substituent” is used herein to refer to an atom radical, a functional group radical or a moiety radical that replaces a hydrogen radical on a molecule. Unless expressly stated otherwise, it should be assumed that any of the substituents may be optionally substituted with one or more groups selected from: alkyl, halogen, haloalkyl, hydroxyalkyl, nitro, amino, hydroxy, cyano, alkylamino, dialkylamino, alkoxy, haloalkoxy, alkoxyalkyl, alkoxyalkoxy, oxo, alkylthio, mercapto, haloalkylthio, aryl, aryloxy, arylthio, heteroaryl, heteroaryloxy, heteroarylthio, acyl, —CO₂-alkyl, —SO₃H, —SO₂NH₂, —SO₂NH-alkyl, —SO₂NH-(alkyl)₂, —CO₂H, —CO₂NH₂, —CO₂NH-alkyl and —CO₂N-(alkyl)₂.

For the purposes of this invention the term “substituted” refers to where a hydrogen radical on a molecule has been replaced by another atom radical, a functional group radical or a moiety radical; these radicals being generally referred to as “substituents.”

The term “protecting group” refers to a group introduced into a molecule to protect a sensitive functional group or specific position on the molecule from reacting when the molecule is exposed to reagents or conditions to transform or react another part of the molecule. Thereafter the protecting group can be removed. Suitable protecting groups are well known in the art and include acid-labile, base-labile, photoremovable, or removable under neutral conditions. See, e.g., Green, Protecting Groups in Organic Synthesis, Wiley 1991, 2^{nd} ed., pp. 309-405, which is incorporated herein by reference. The term “amine protecting group” refers to a moiety capable of protecting an amine functional group from reacting. Exemplary amine protecting groups for the present invention include acyl groups (such as acetyl), t-butoxycarbonyl (t-BOC), benzyloxycarbonyl, trifluoroacetyl, CH₃OC(O)—, EtOC(O)—, Fmoc, Troc, Phenoc, Teoc and PhC(O)— groups, and forming cyclicimdes (e.g. phthalimide, maleimide, 2,3-dichloromaleimide, succinimide and dihydrophthalimide) and pyrroles (e.g. dimethylpyrrole).

Cyclicimides are useful protecting groups for masking primary amines. They are formed by reacting the primary amine to be masked with a reagent such as phthalic anhydride or maleamic anhydride, thereby incorporating the amine into the cyclicimide, as shown below.

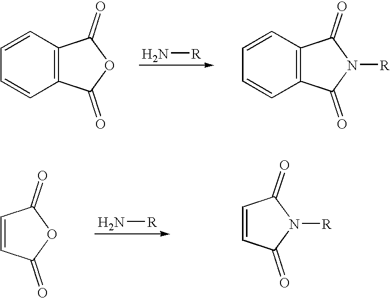

Thereafter, the cyclicimides can be cleaved under a variety of conditions to give the primary amine in good yield. See Green at pp. 358-359. 2,5-Dimethylpyrrole operates similarly.

The term “cancer” refers to any malignant growth or tumor caused by abnormal and uncontrolled cell division. It may spread to other parts of the body through the lymphatic system or the blood stream. For the purposes of the method of treating cancer described in this application, cancer includes lymphatic cancer, breast cancer, ovarian cancer, epidermoid tumors, colon cancer, prostate cancer, kidney cancer, bladder cancer, larynx cancer, esophagus cancer, stomach cancer, and lung cancer.

The compounds synthesized by this invention may contain an asymmetric carbon atom and may thus give rise to stereoisomers, such as enantiomers and diastereomers. The stereioisomers of the instant invention are named according to the Cahn-Ingold-Prelog System. While shown without respect to stereochemistry in formula (I), the present invention includes the synthesis of all the individual possible stereoisomers; as well as the racemic mixtures and other mixtures of R and S stereoisomers (scalemic mixtures which are mixtures of unequal amounts of enantiomers). It should be noted that stereoisomers of the invention having the same relative configuration at a chiral center may nevertheless have different R and S designations depending on the substitution at the indicated chiral center.

This invention is also directed to pharmaceutical compositions containing a therapeutically effective amount of a compound selected from (E)N-{4-[3-chloro-4-fluoroanilino]-3-cyano-7-ethoxy-6-quinolinyl}-4-(methylamino)-2-butenamide, (E)N-{4-[3-chloro-4-(2-pyridinylmethoxy)anilino]-3-cyano-7-ethoxy-6-quinolinyl}-4-(methylamino)-2-butenamide, (E)N-{4-[3-chloro-4-(3-fluorobenzyloxy)anilino]-3-cyano-7-ethoxy-6-quinolinyl}-4-(methylamino)-2-butenamide and (E)N-{4-[(3-chloro-4-benzyloxy)anilino]-3-cyano-7-ethoxy-6-quinolinyl}-4-(methylamino)-2-butenamide, and a pharmaceutically acceptable carrier. In a preferred embodiment of the composition of the present invention the compound is (E)N-{4-[3-chloro-4-(2-pyridinylmethoxy)anilino]-3-cyano-7-ethoxy-6-quinolinyl}-4-(methylamino)-2-butenamide.

The pharmaceutically acceptable carrier contained in the composition of the present invention may be for example a diluent, an aerosol, a topical carrier, an aqueous solution, a nonaqueous solution or a solid carrier. The carrier may be a polymer or a toothpaste. A carrier in this invention encompasses any of the standard pharmaceutically accepted carriers, such as phosphate buffered saline solution, acetate buffered saline solution, water, emulsions such as an oil/water emulsion or a triglyceride emulsion, various types of wetting agents, tablets, coated tablets and capsules. The compositions of the present invention may be formulated with conventional excipients, such as a filler, a disintegrating agent, a binder, a lubricant, a flavoring agent or a color additive.

When provided orally or topically, such compounds would be provided to a subject by delivery in different carriers. Typically, such carriers contain excipients such as starch, milk, sugar, certain types of clay, gelatin, stearic acid, talc, vegetable fats or oils, gums, or glycols. The specific carrier would need to be selected based upon the desired method of delivery, for example, phosphate buffered saline (PBS) could be used for intravenous or systemic delivery and vegetable fats, creams, salves, ointments or gels may be used for topical delivery.

The compounds of the present invention may be delivered together with suitable diluents, preservatives, solubilizers, emulsifiers, adjuvants and/or carriers useful in treatment or prevention of neoplasm. Such compositions are liquids or lyophilized or otherwise dried formulations and include diluents of various buffer content (for example, Tris-HCl, acetate, phosphate), pH and ionic strength, additives such as albumins or gelatin to prevent absorption to surfaces, detergents (for example, TWEEN 20, TWEEN 80, PLURONIC F68, bile acid salts), solubilizing agents (for example, glycerol, polyethylene glycerol), anti-oxidants (for example ascorbic acid, sodium metabisulfate), preservatives (for example, thimerosal, benzyl alcohol, parabens), bulking substances or tonicity modifiers (for example, lactose, mannitol), covalent attachment of polymers such as polyethylene glycol, complexation with metal ions, or incorporation of the compound into or onto particulate preparations of hydrogels or liposomes, micro-emulsions, micelles, unilamellar or multilamellar vesicles, erythrocyte ghosts, or spheroblasts. Such compositions will influence the physical state, solubility, stability, rate of in vivo release, and rate of in vivo clearance of the compound or composition. The choice of compositions will depend on the physical and chemical properties of the compound capable of treating or preventing a neoplasm.

The compounds of the present invention may be delivered locally via a capsule that allows a sustained release of the compound over a period of time. Controlled or sustained release compositions include formulation in lipophilic depots (for example, fatty acids, waxes, oils).

The present invention further provides a method of using the compounds disclosed herein, (E)N-{4-[3-chloro-4-fluoroanilino]-3-cyano-7-ethoxy-6-quinolinyl}-4-(methylamino)-2-butenamide, (E)N-{4-[3-chloro-4-(2-pyridinylmethoxy)anilino]-3-cyano-7-ethoxy-6-quinolinyl}-4-(methylamino)-2-butenamide, (E)N-{4-[3-chloro-4-(3-fluorobenzyloxy)anilino]-3-cyano-7-ethoxy-6-quinolinyl}-4-(methylamino)-2-butenamide and (E)N-{4-[(3-chloro-4-benzyloxy)anilino]-3-cyano-7-ethoxy-6-quinolinyl}-4-(methylamino)-2-butenamide, as active therapeutic substances for preventing or inhibiting cancer.

The present invention further provides a method of treating cancer in humans, which comprises administering to the infected individual an effective amount of a compound or a pharmaceutical composition of the invention. A “therapeutically effective amount” is an amount sufficient to cure or ameliorate symptoms of a cancer. The effective dosage of active ingredient employed may vary depending on the particular compound employed, the mode of administration and the severity of the condition being treated. In general, satisfactory results are obtained when the compounds of the invention are administered at a daily dosage of from about 0.5 to about 1000 mg/kg of animal body weight, optionally given in divided doses two to four times a day, or in sustained release form. For most large mammals the total daily dosage is from about 1 to 1000 mg, preferably from about 2 to 500 mg. Dosage forms suitable for internal use comprise from about 0.5 to 1000 mg of the active compound in intimate admixture with a solid or liquid pharmaceutically acceptable carrier. This dosage regimen may be adjusted to provide the optimal therapeutic response. For example, several divided doses may be administered daily or the dose may be proportionally reduced as indicated by the exigencies of the therapeutic situation.

The compounds of the present invention may be delivered alone or in combination with other compounds used to treat cancer or with radiation therapy. Such compounds include but are not limited to imatinib mesylate (GLEEVEC), hydroxyurea, IFN-\acute{α}, cytotoxic agents, NSAIDS, gemcitabine, EGFR inhibitors, MEK inhibitors, farnesyltransferase, taxol, vinblastine, cisplatin, cyclophosamide5-fluorouracil, adriamycin, bleomycin, etoposide, campptothecin, tamoxifen or wortmaninin.

In a preferred embodiment of the method of treating cancer disclosed in the present application, the cancer being treated is selected from breast cancer, ovarian cancer, epidermoid tumors, colon cancer, prostate cancer, kidney cancer, bladder cancer, larynx cancer, esophagus cancer, stomach cancer, and lung cancer. In another preferred embodiment the cancer being treated is breast cancer or ovarian cancer.

In a preferred embodiment of this method of synthesizing the compounds of formula (I) by deprotecting a compound of formula (II), further comprises the step of forming the compound of formula (II) by coupling an anilinoquinoline of formula (III): 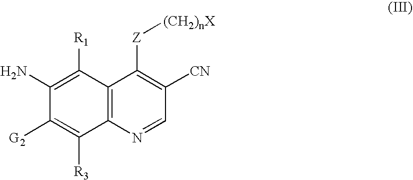
with an acid of formula (VII): 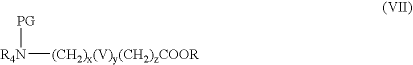
to form a compound of formula (II), wherein R is H, and R₁, R₃, R₄, Z, G₂, V, PG, n, x, y, and z are as previously defined.

In yet another preferred embodiment of this method of synthesizing the compounds of formula (I), further comprises the step of forming the anilinoquinoline of formula (III) by:

a. arylating a compound of formula (V): 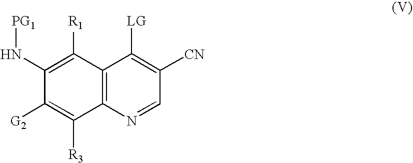
with a compound of formula HZ-(CH₂)ₙX to form an intermediate of formula (IV):

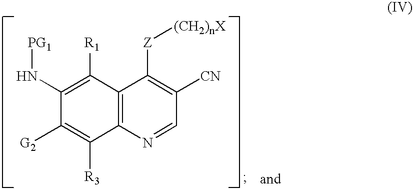

b. deprotecting the intermediate of formula (IV) to obtain the anilinoquinoline compound of formula (III);
wherein LG is selected from the group of halo, mesylate, tosylate and trifylate, PG₁ is an amine protecting group, and wherein R₁, R₃, R₄, Z, G₂, PG, and n are as previously defined.

Another embodiment of the method of preparing compounds of formula (I) is where the method also involves the step of forming a compound of formula (II) by coupling a compound of formula (III) with an acid of formula (VII), using a coupling reagent selected from DCC, benzotriazalyloxytrispyrrolidinophosphonium hexafluorophosphate (PyBOP), or N-ethyl-(N′-3-dimethylaminopropyl)-carbodiimide.HCl (EDCI) with 1-hydroxybenzotriazole (HOBt), though DCC is the most preferred coupling reagent. It is further preferred that this method also comprise the steps of arylating the 4-position of a compound of formula (V) with a reagent of formula HZ-(CH₂)ₙX, preferably using methylsulfonic acid, to form an intermediate of formula (IV), which is subsequently deprotected to yield a compound of formula (III). Wherein LG is a leaving group, such as halo, mesylate, tosylate or triflate, with Cl being the most preferred, and PG₁ is an amine protecting group, with exemplary groups being an acyl group (such as acetyl), t-butoxycarbonyl (t-BOC), CH₃OC(O)—, EtOC(O)—, Fmoc, Troc, Phenoc, Teoc, trifluoroacetyl, benzoxy carbonyl, PhC(O)—, 2,5-dimethylpyrrole, phthalimide, 2,3-dichloromaleimide, succinimide, dihydrophthalimide or maleimide. It is also preferable that PG₁ be removed using an acid. One skilled in the art would also know of other suitable leaving groups, which could be utilized. The more preferred protecting groups are acetyl, t-BOC, trifluoroacetamide, benzamide, 2,5-dimethylpyrrole, phthalimide and maleimide, with t-BOC and acetyl being the most preferred.

Another embodiment of the methods of preparing a compound of formula (I) is where the compounds are limited by the following provisos:

(i) when R₆ is alkenyl of 2-7 carbon atoms or alkynyl of 2-7 carbon atoms, such alkenyl or alkynyl moiety is bound to a nitrogen or oxygen atom through a saturated carbon atom;

(ii) when Y is —NR₆— and R₇ is —NR₆R₆, —N(R₆)₃.N(R₈)₃⁺, or —NR₆(OR₆), then g=2-6;

(iii) when M is —O— and R₇ is —OR₆ then p=1-4;

(iv) when Y is —NR₆— then k=2-4;

(v) when Y is —O— and M or W is —O— then k=1-4;

(vi) when W is not a bond with Het bonded through a nitrogen atom then q=2-4;
when W is a bond with Het bonded through a nitrogen atom and Y is —O— or —NR₆— then k=2-4; and
provided that L can be an unsubstituted phenyl ring only when m>0 and T is not —CH₂NH— or —CH₂O—.

Another embodiment of the inventive methods of preparing compounds of formula (I) is wherein X is cycloalkyl, which may be optionally substituted with one or more alkyl groups, or is a pyridinyl, pyrimidinyl, or phenyl ring; wherein the pyridinyl, pyrimidinyl, or phenyl ring may be optionally mono- di-, or tri-substituted as described previously, or X is a radical having the formula: 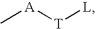
wherein A, T and L are as defined previously, and R₁, G₂, and R₃ are each, independently, hydrogen, halogen, alkyl of 1-6 carbon atoms, alkenyl of 2-6 carbon atoms, alkynyl of 2-6 carbon atoms, alkenyloxy of 2-6 carbon atoms, alkynyloxy of 2-6 carbon atoms, hydroxymethyl, halomethyl, alkanoyloxy of 1-6 carbon atoms, alkenoyloxy of 3-8 carbon atoms, alkynoyloxy of 3-8 carbon atoms, alkanoyloxymethyl of 2-7 carbon atoms, alkenoyloxymethyl of 4-9 carbon atoms, alkynoyloxymethyl of 4-9 carbon atoms, alkoxymethyl of 2-7 carbon atoms, alkoxy of 1-6 carbon atoms, alkylthio of 1-6 carbon atoms, alkylsulphinyl of 1-6 carbon atoms, alkylsulphonyl of 1-6 carbon atoms, alkylsulfonamido of 1-6 carbon atoms, alkenylsulfonamido of 2-6 carbon atoms, alkynylsulfonamido of 2-6 carbon atoms, hydroxy, trifluoromethyl, trifluoromethoxy, cyano, nitro, carboxy, carboalkoxy of 2-7 carbon atoms, carboalkyl of 2-7 carbon atoms, phenoxy, phthalimide, phenyl, thiophenoxy, benzyl, amino, hydroxyamino, alkoxyamino of 1-4 carbon atoms, alkylamino of 1-6 carbon atoms, dialkylamino of 2 to 12 carbon atoms, N-alkylcarbamoyl, N,N-dialkylcarbamoyl, N-alkyl-N-alkenylamino of 4 to 12 carbon atoms, or N,N-dialkenylamino of 6-12 carbon atoms.

In another embodiment of the methods of preparing compounds of formula (I) the compounds are defined by X being cycloalkyl, which may be optionally substituted with one or more C₁-C₆-alkyl groups, or is a pyridinyl, pyrimidinyl, or phenyl ring; wherein the pyridinyl, pyrimidinyl, or phenyl ring may be optionally mono- di-, or tri-substituted with the groups described previously. Z is preferably NR^{z′}. When Z is NR^{z′} it is preferable that X be an aromatic moiety, such as pyridinyl, pyrimidinyl, or phenyl ring, with phenyl being the most preferred. These aromatic moieties may be mono-, bi-, or tri-substituted. When Z is NR^{z′} it is also preferable that z and n are 0, y is 1, V is ethylene and R₁, G₂, and R₃ are each, independently, hydrogen, halogen, alkyl of 1-6 carbon atoms, halomethyl, alkoxy of 1-6 carbon atoms, hydroxy, trifluoromethyl, trifluoromethoxy, cyano, nitro, phenoxy, phthalimide, phenyl, thiophenoxy, benzyl, amino, alkylamino of 1-6 carbon atoms, or dialkylamino of 2 to 12 carbon atoms. In this embodiment when X is phenyl and Z is NR^{z′} it is further preferable that R₁, G₂, and R₃ be limited to hydrogen, halogen, alkyl, alkoxy, hydroxy, trifluoromethyl, trifluoromethoxy and that X is only optionally substituted with halo, alkyl, trifluoromethyl and alkoxy. It is also preferable that when X is phenyl and Z is NR^{z′} that R₄ is methyl, ethyl, propyl or isopropyl, R^{z′} is H and that R₁, G₂, and R₃ are further limited to hydrogen, halogen, methoxy, ethoxy, hydroxy, trifluoromethyl, or trifluoromethoxy. A specific embodiment is where the compound prepared is (E)N-{4-[3-chloro-4-fluoroanilino]-3-cyano-7-ethoxy-6-quinolinyl}-4-(methylamino)-2-butenamide.

Another embodiment of the methods of preparing the compounds of formula (I) is where the compounds are defined by X being a radical having the formula: 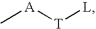
where A, T and L are as defined previously. A is most preferably an optionally mono- or di-substituted phenyl ring, and when A is a phenyl ring it is preferable that T is a tether containing an ether or thio linkage, though an ether linkage is most preferred. Z is preferably NR^{z′} and L is preferably an optionally mono- or di-substituted 5- or 6-membered hetroaryl, such as pyridine, pyrimidine, pyriazine, or pyrazine. The amide moiety at the 6-position of the quinoline ring is preferably limited to where z and n are 0, y is 1 and V is ethylene, while R₁, G₂, and R₃ are preferably hydrogen, halogen, alkyl of 1-6 carbon atoms, halomethyl, alkoxy of 1-6 carbon atoms, hydroxy, trifluoromethyl, trifluoromethoxy, cyano, nitro, phenoxy, phthalimide, phenyl, thiophenoxy, benzyl, amino, alkylamino of 1-6 carbon atoms, or dialkylamino of 2 to 12 carbon atoms. When T is an ether linkage it is preferable that m is 1 and that A is optionally mono- or di-substituted with a substituent selected from the group consisting of halogen, alkyl of 1-6 carbon atoms, halomethyl, alkoxy of 1-6 carbon atoms, trifluoromethyl, cyano, amino, alkylamino of 1-6 carbon atoms, and dialkylamino of 2 to 12 carbon atoms. When T is —OCH₂— it is preferable that R₄ is methyl, ethyl, propyl or isopropyl, R^{z′} is H, L is pyridine, A and L are optionally mono- or di-substituted with a substituent selected from the group consisting of halogen, methyl, ethyl, methoxy, ethoxy, and trifluoromethyl, and that R₁, G₂, and R₃ are further limited to hydrogen, halogen, methoxy, ethoxy, hydroxy, trifluoromethyl, or trifluoromethoxy. A specific embodiment is where the compound prepared is (E) N-{4-[3-Chloro-4-(2-pyridinylmethoxy)anilino]-3-cyano-7-ethoxy-6-quinolinyl)-4-(methylamino)-2-butenamide.

Another embodiment of the methods of preparing the compounds of formula (I) is also where the compounds are defined by X being a radical having the formula: 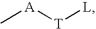
wherein A and L are phenyl rings, Z is NR^{z′} and T is a tether containing an ether or thio linkage, though an ether linkage is most preferred. The amide moiety at the 6-position of the quinoline ring is preferably limited to where z and n are 0, y is 1 and V is ethylene, while R₁, G₂, and R₃ are preferably hydrogen, halogen, alkyl of 1-6 carbon atoms, halomethyl, alkoxy of 1-6 carbon atoms, hydroxy, trifluoromethyl, trifluoromethoxy, cyano, nitro, phenoxy, phthalimide, phenyl, thiophenoxy, benzyl, amino, alkylamino of 1-6 carbon atoms, or dialkylamino of 2 to 12 carbon atoms. When T is an ether linkage it is preferable that m is 1 and that A is optionally mono- or di-substituted with a substituent selected from the group consisting of halogen, alkyl of 1-6 carbon atoms, halomethyl, alkoxy of 1-6 carbon atoms, trifluoromethyl, cyano, amino, alkylamino of 1-6 carbon atoms, and dialkylamino of 2 to 12 carbon atoms. When T is —OCH₂— it is preferable that R₄ is methyl, ethyl, propyl or isopropyl, R^{z′} is H, A is optionally mono- or di-substituted and L is optionally mono-, di- or tri-substituted with a substituent selected from the group consisting of halogen, methyl, ethyl, methoxy, ethoxy, and trifluoromethyl, and that R₁, G₂, and R₃ are further limited to hydrogen, halogen, methoxy, ethoxy, hydroxy, trifluoromethyl, or trifluoromethoxy. Specific embodiments are where the compound prepared is (E)N-{4-[(3-Chloro-4-benzyloxy)anilino]-3-cyano-7-ethoxy-6-quinolinyl}-4-(methylamino)-2-butenamide or (E)N-{4-[3-Chloro-4-(3-fluorobenzyloxy)anilino]-3-cyano-7-ethoxy-6-quinolinyl}-4-(methylamino)-2-butenamide.

Another embodiment of the method of preparing compounds of formula (I) is where the method also involves the step of coupling a compound of formula (III) with an acid of formula (VII″), using a coupling reagent selected from DCC, benzotriazalyloxytrispyrrolidinophosphonium hexafluorophosphate (PyBoP), or N-ethyl-(N′-3-dimethylaminopropyl)-carbodiimide.HCl (EDCI) with 1-hydroxybenzotriazole (HOBt), though DCC is the most preferred coupling reagent. Alternatively, the acid of formula (VII″) can be convert to the corresponding acid halide, for example the acid chloride, and then coupled to the aniline compound of formula (III). The art is replete with methods for converting carboxylic acids into the corresponding acid halides using reagents such as SOCl₂ and oxalyl chloride. The coupling using the coupling reagent is the preferred method. It is further preferred that this method also comprise the steps of arylating the 4-position of a compound of formula (V) with a reagent of formula HZ-(CH₂)ₙX, preferably using methylsulfonic acid, to form an intermediate of formula (IV), which is subsequently deprotected to yield a compound of formula (III). Wherein LG is a leaving group, such as halo, mesylate, tosylate or triflate, with Cl being the most preferred, and PG₁ is an amine protecting group, with exemplary groups being an acyl group (such as acetyl), t-butoxycarbonyl (t-BOC), CH₃OC(O)—, EtOC(O)—, Fmoc, Troc, Phenoc, Teoc, trifluoroacetyl, benzoxy carbonyl, PhC(O)—, 2,5-dimethylpyrrole, phthalimide, 2,3-dichloromaleimide, succinimide, dihydrophthalimide or maleimide. It is also preferable that PG₁ be removed using an acid. One skilled in the art would also know of other suitable leaving groups, which could be utilized. The more preferred protecting groups are acetyl, t-BOC, trifluoroacetamide, benzamide, 2,5-dimethylpyrrole, phthalimide and maleimide, with t-BOC and acetyl being the most preferred.

A embodiment of the method of preparing acid compounds of formula (VII) is wherein the method further comprises the steps of alkylating a compound of formula (VI) with a primary amine of the formula NH₂R₄ to give an aminoester intermediate and subsequently protecting the aminoester intermediate by alkyating on a protecting group to form an ester compound of formula (VII′). In this embodiment is also preferable that the compounds of formula (VII) be defined by z being 0, y being 1 and V being ethylene. More preferably R₄ is methyl, ethyl, propyl or isopropyl. A specific embodiment is where the acid compound of formula (VII) is

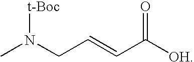

The preferred compounds synthesized by the present invention include:
(E)N-{4-[3-chloro-4-fluoroanilino]-3-cyano-7-ethoxy-6-quinolinyl}-4-(methylamino)-2-butenamide;
(E)N-{4-[3-chloro-4-(2-pyridinylmethoxy)anilino]-3-cyano-7-ethoxy-6-quinolinyl}-4-(methylamino)-2-butenamide;
(E)N-{4-[3-chloro-4-(3-fluorobenzyloxy)anilino]-3-cyano-7-ethoxy-6-quinolinyl}-4-(methylamino)-2-butenamide; and
(E)N-{4-[(3-chloro-4-benzyloxy)anilino]-3-cyano-7-ethoxy-6-quinolinyl}-4-(methylamino)-2-butenamide.
   General Synthesis

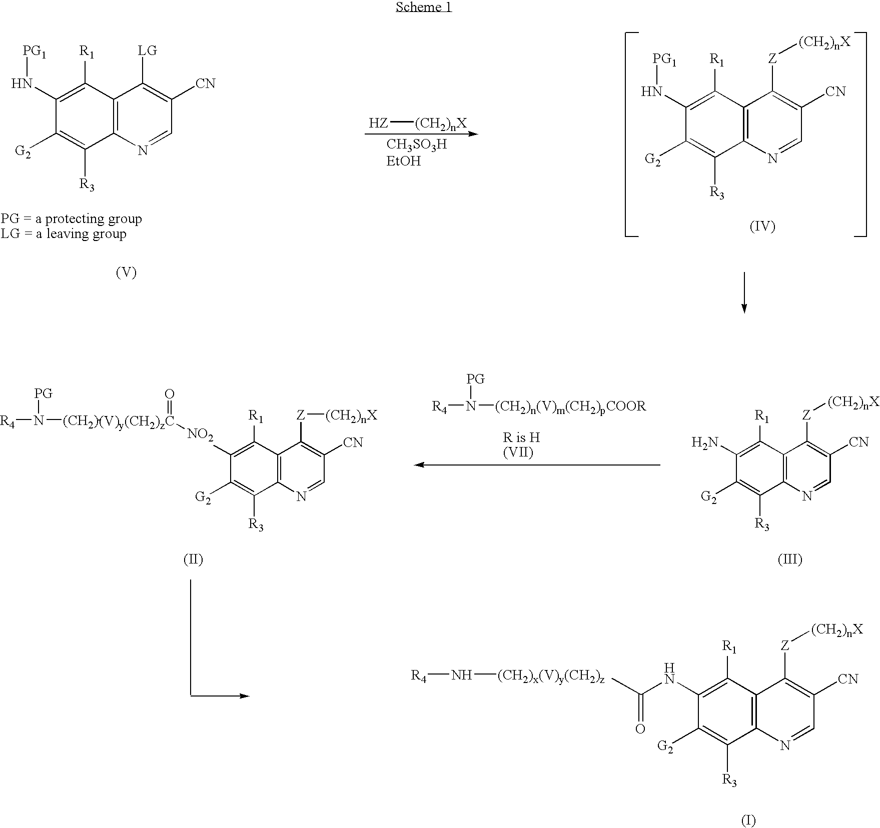

Scheme 1 illustrates the synthesis of a des-alkylaminoquinoline of formula (I) from the starting protected anilinoquinoline of formula (V), wherein the protecting groups can be any one of those usually employed to protect an amine, such as an acyl group (such as acetyl), t-butoxycarbonyl (t-BOC), benzyloxycarbonyl, CH₃OC(O)—, EtOC(O)—, Fmoc, Troc, Phenoc, Teoc, PhC(O)— groups, cyclicimdes (e.g. phthalimide, maleimide, 2,3-dichloromaleimide, succinimide and dihydrophthalimide) or a pyrrole (e.g. dimethylpyrrole). The protected anilinoquinoline is first reacted with methylsulfonic acid and a reagent of formula HZ-(CH₂)ₙX, wherein Z can be NR^{z′}, O or S and R^{z′} is H or alkyl, n is 0 or 1 and X is as previously defined, to form the intermediate of formula (IV). Reagents such as pyridinium hydrochloride, HCl, sulfuric acid, trifluoroacetic acid, and the like, can be used in place of methylsulfonic acid. The intermediate is then deprotected to provide the anilinoquinoline of formula (III).

The anilinoquinoline of formula (III) is then coupled with an acid of formula (VII) using a standard coupling reagent such as DCC, benzotriazalyloxytrispyrrolidinophosphonium hexafluorophosphate (PyBoP), or N-ethyl-(N′-3-dimethylaminopropyl)-carbodiimide.HCl (EDCI) with 1-hydroxybenzotriazole (HOBt). The technical literature is replete with other coupling reagents and one skilled the art would be aware of such reagents. DCC is preferable. The acid of formula (VII) is defined by V being an ethylene or an acetylene group, x is 0, 1, 2, or 3, y is 0 or 1, z is 0, 1, 2 or 3, R is H, R₄ is an alkyl group of 1 to 6 carbon atoms and PG is an amine protecting group that is stable to basic conditions, such as an acyl group (such as acetyl), t-butoxycarbonyl (t-BOC), benzyloxycarbonyl, CH₃OC(O)—, EtOC(O)—, Fmoc, Troc, Phenoc, Teoc or PhC(O)—. The most preferred is t-BOC. This is not intended to be an exhaustive list and one skilled in the art would be aware of other suitable protecting groups. The synthesis of such acid compounds is shown in Scheme 2 below. This coupling reaction produces compounds of formula (II).

Amidoquinolines of formula (II) can then be deprotected under acid or neutral conditions to provide the des-alkyl metabolites of formula (I). If PG is a t-BOC group then the acid is preferably HCl, though other acids are also suitable.

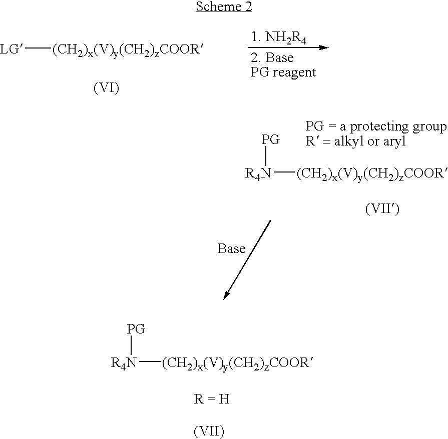

As illustrated in Scheme 2, esters of formula (VI), wherein LG′ is a leaving group, such as halo, mesylate, tosylate or triflate, and R′ is alkyl or aryl, are reacted first with primary amines and second with reagent that alkylates a protecting group on the amine nitrogen to give protected alkylamino esters of formula (VII′). Halogens are the preferred leaving group, especially bromine. The protecting group can be any of the commonly used amine protecting groups that are stable to basic conditions, such as an acyl group (such as acetyl), t-butoxycarbonyl (t-BOC), benzyloxycarbonyl, CH₃OC(O)—, EtOC(O)—, Fmoc, Troc, Phenoc, Teoc or PhC(O)—. Tert-butoxycarbonyl is the most preferred, and (BOC)₂O is the most preferred reagent for installing the protecting group. Saponificaton of the esters provides the corresponding acids of formula (VII), wherein R is H. Coupling of the acids with substituted 4-anilinoquinolines followed by deprotection provides the des-methyl compounds of formula (I) as shown in Scheme 1.
Specific Synthesis

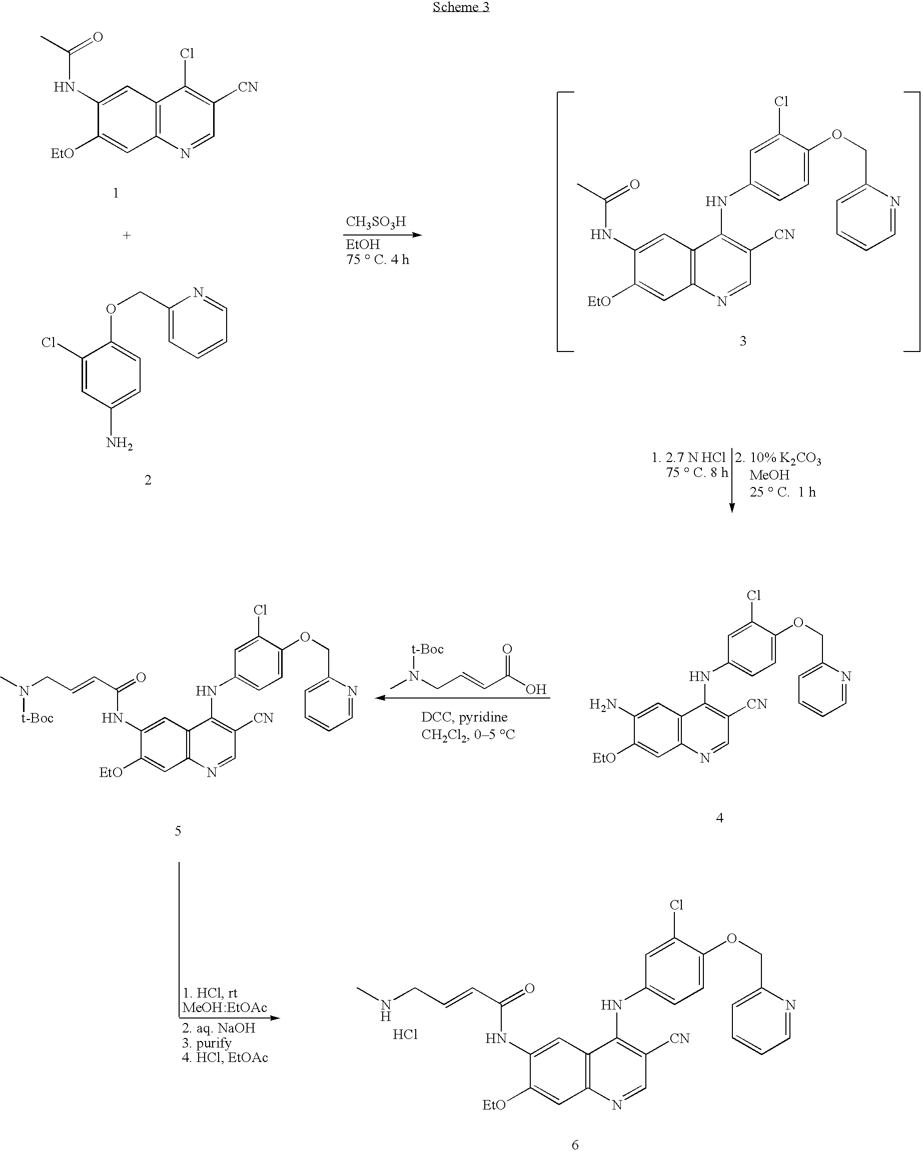

In Scheme 3 a specific synthesis of 6-(4-alkylamino)-2-butenamido quinoline 6 is illustrated. Compound 1 is arylated with compound 2 using methylsulfonic acid in ethanol at about 75° C. to give intermediate 3. This intermediate is then deprotected under acidic conditions using approximately 2.7 M HCl and heating at 75° C. A basic work up with potassium carbonate then affords 6-anilinoquinoline 4.

Compound 4 is then coupled with t-BOC protected 4-methylaminocrotonic acid using DCC and pyridine at about ice bath temperatures to yield compound 5. Compound 5 is subsequently deprotected using mild HCl conditions and a basic work up to provide the free base of 6-(4-alkylamino)-2-butenamido quinoline 6. After purification, the corresponding HCl salt can be formed by exposing the compound to HCl in ethylacetate.

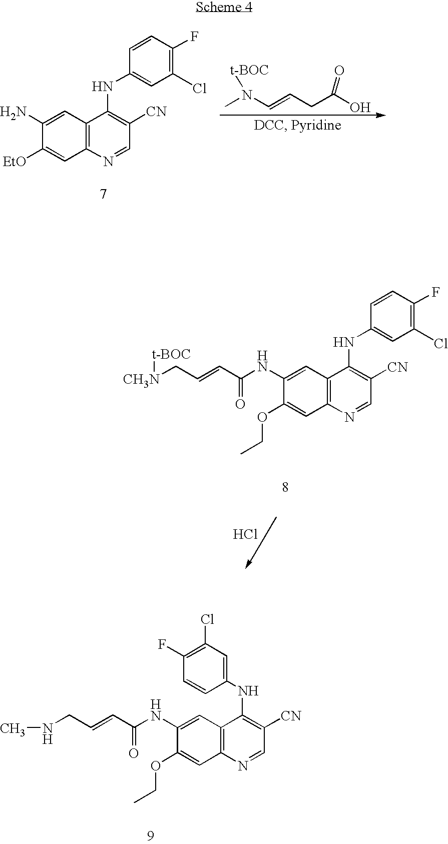

Scheme 4 shows the synthesis of 6-(4-alkylamino)-2-butenamidoquinoline 9. Compound 7 was formed analogous to compound 4 in Scheme 3, substituting 3-chloro-4-fluoroaniline for 3-chloro-4-(2-pyridinylmethoxy)aniline. Compound 7 is then coupled with t-BOC protected 4-methylaminocrotonic acid using DCC and pyridine to give compound 8, which is subsequently deprotected using HCl to provide 6-(4-alkylamino)-2-butenamidoquinoline 9.

The following are examples prepared using the methods illustrated in the above schemes. These examples are only some of the compounds envisioned by the present invention and should not be viewed as limiting the present invention in any way.

### EXAMPLE 1

### 4-[N-(t-butyloxycarbonyl)methylamino]crotonic acid

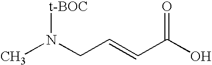

### Preparation of methyl 4-methylamino-crotonate HCl

A solution of methyl 4-bromocrotonate (66 mL, 0.56 mol) in THF (500 mL) and a solution of 2.0 M methylamine in THF (700 mL, 1.4 mol) were added through additional funnel simultaneously at −20° C. over 30 min. The reaction mixture was a light yellow suspension. After addition, the reaction mixture was stirred at −5° C. for 2 h, then filtered and washed with THF (2×30 mL). The filtrate was concentrated in vacuo to give a brown oil. A solution of HCl in IPA (10.5%, 200 mL) was added to the brown oil through an additional funnel at 0-5° C. The reaction mixture was stirred for 30 min, and then concentrated in vacuo to give brown red oil. CH₂Cl₂ (˜20 mL) was added and the solution placed in the refrigerator (˜5° C.) to allow for crystallization. The mother liquor gave several batches of crystals. The combined batches were recrystallized from CH₂Cl₂ to obtain 17 g (22% yield) of the desired compound as a colorless solid. ¹H NMR (CDCl₃, ppm) δ 7.02 (m, 1H), 6.28 (d, 1H), 3.80 (s, 2H), 3.77 (s, 3H), 2.70 (t, 3H); ¹³C NMR (DMSO-d₆, ppm) δ 165.11, 138.39, 125.43, 51.62, 47.69, 31.81; IR (KBr): νₘₐₓ 3426, 3285, 3037, 2954, 2805, 2423, 1719, 1667, 1438, 1348, 1272, 1209, 1158, 1027, 1000 cm⁻¹; Anal. Calc'd for C₆H₁₂ClNO₂: C, 43.51; H, 7.30; N, 8.46. Found: C, 43.42; H, 7.36; N, 8.37.

### Preparation of methyl 4-[N-(t-butyloxycarbonyl)methylamino]crotonate

To a suspension of methyl 4-methylamino-crotonate (14.1 g, 85.3 mmol) in CH₂Cl₂ (150 mL) were added DMAP (10.4 g, 85.3 mmol) and triethylamine (25 mL, 179.2 mmol) while cooling in an ice bath. To this suspension (Boc)₂O (22.4 g, 102.4 mmol) in CH₂Cl₂ (50 mL) was added slowly. The reaction mixture was stirred at room temperature for 19 h under N₂, then quenched with H₂O (150 mL). The organic layer was washed with H₂O (2×150 mL), 3 N HCl (200 mL), followed by H₂O (100 mL). The organic layer was dried over Na₂SO₄, filtered and concentrated in vacuo to give 17.3 g (89% yield) of N-Boc Methyl Crotonate as a brown oil. ¹H NMR (CDCl₃, ppm) δ 6.87 (m, 1H), 5.84 (d, 1H), 3.99 (bs, 2H, NCH₂), 3.75 (s, 3H), 2.85 (bs, 3H), 1.46 (s, 9H); ¹³C NMR (CDCl₃, ppm) δ 166.51, 155.38, 143.91, 121.37, 79.98, 51.61, 49.64, 34.36, 28.55; IR (KBr): νₘₐₓ 3591, 3441, 2976, 2953, 1726, 1699, 1480, 1452, 1392, 1366, 1276, 1228, 1169, 1148, 1039, 947 cm⁻¹; Anal. Calc'd for C₁₁H₁₉NO₄: C, 57.62; H, 8.35; N, 6.11. Found: C, 57.86; H, 8.57; N, 6.30.

### Preparation of 4-[N-(t-butyloxycarbonyl)methylamino]-crotonic acid

To a solution of methyl 4-[N-(t-butyloxycarbonyl)methylamino]-crotonate (17.5 g, 76.3 mmol) in THF (200 mL) cooled in an ice bath, KOH (17.1 g, 305 mmol) in H₂O (100 mL) was added through an additional funnel. The reaction mixture was stirred in the ice bath for 15 min before removed to stir at room temperature under N₂. After 16 h, the reaction mixture was concentrated in vacuo to give an aqueous layer. The aqueous layer was washed with CH₂Cl₂ (2×60 mL) to remove organic impurities, then acidified to pH=1˜2 with 3 N HCl (˜130 mL). The aqueous layer was extracted with CH₂Cl₂ (4×80 mL). The combined organic layer was dried over Na₂SO₄, filtered and concentrated in vacuo to give 16.0 g (97.5% yield) of the titled compound as a light yellow oil. ¹H NMR (CDCl₃, ppm) δ 6.98 (m, H), 5.85 (d, 1H), 3.99 (bs, 2H), 2.87 (bs, 3H), 1.46 (s, 9H); ¹³C-NMR (CDCl₃, ppm) δ 170.34, 155.72, 145.60, 121.44, 80.18, 49.70, 34.47, 28.23; IR (KBr): νₘₐₓ 3417, 3144, 2977, 2934, 1699, 1482, 1455, 1394, 1368, 1252, 1152, 948 cm⁻¹; Anal. Calcd for C₁₀H₁₇NO₄: C, 55.80; H, 7.96; N, 6.51. Found: C, 54.46; H, 8.10; N, 6.32.

### EXAMPLE 2

### (E)N-{4-[3-chloro-4-fluoroanilino]-3-cyano-7-ethoxy-6-quinolinyl}-4-[N-(t-butyloxycarbonyl)methylamino]-2-butenamide

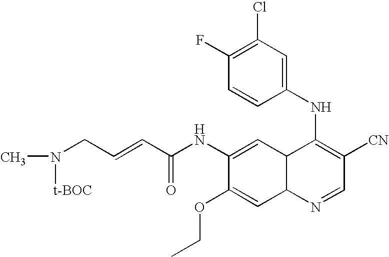

### Coupling of N-Boc Crotonic Acid with 4-(3-chloro-4-fluoro-phenylamino)-3-cyano-7-ethoxy-quinoline

To a solution of 4-[N-(t-butyloxy carbonyl)methylamino]-crotonic acid (16.0 g, 74.4 mmol) in CH₂Cl₂ (150 mL), cooled in an ice bath, were added pyridine (60 mL, 744 mmol) and DCC (15.3 g, 74.4 mmol). A suspension of 4-(3-chloro-4-fluoro-phenylamino)-3-cyano-7-ethoxy-quinoline (13.3 g, 37.2 mmol) in CH₂Cl₂ (100 mL) was added slowly. The reaction flask was covered by alumina foil, stirred at rt under N₂. After 3 d, the reaction mixture was diluted with CH₂Cl₂ (120 mL), then filtered. The filtrate was washed with H₂O (2×60 mL), 2 N HCl (2×60 mL), and H₂O (60 mL). The organic layer was dried over Na₂SO₄, filtered and concentrated in vacuo to give crude product as a brown red residue. Purification via SiO₂, eluted with EtOAc to give 7.5 g of 6-(4-[N-(t-butyloxycarbonyl)-methylamino]-but-2-enyl)-4-(3-chloro-4-fluoro-phenylamino)-3-cyano-7-ethoxy-quinoline. Recrystallization from ethyl acetate gave 4.9 g (12% yield) of the compound as a light yellow solid. ¹H NMR (CDCl₃, ppm) δ 9.17 (s, 1H), 8.46 (s, 1H), 7.99 (s, 1H), 7.19-6.96 (m, 3H), 6.83 (m, 1H), 6.05 (d, 1H), 4.25 (q, 2H), 4.02 (bs, 2H), 2.85 (bs, 3H), 1.53 (t, 3H), 1.47 (s, 9H); IR (Nujol): νₘₐₓ 3313, 2951, 2855, 2218, 1707, 1679, 1640, 1531, 1502, 1458, 1377, 1273, 1164, 1146, 1042, 869, 855, 774 cm⁻¹; m.p.: 106.5-108.0° C.; Anal. Calcd for C₂₈H₂₉N₅O₄ClF: C, 60.76; H, 5.24; N, 12.66. Found: C, 59.70; H, 5.89; N, 10.83.

### EXAMPLE 3

### (E)N-{4-[3-chloro-4-fluoroanilino]-3-cyano-7-ethoxy-6-quinolinyl}-4-(methylamino)-2-butenamide

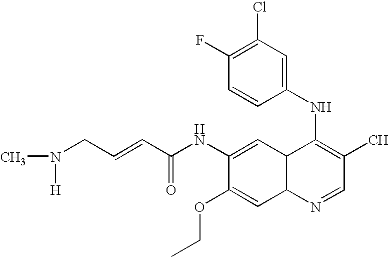

### Hydrolysis of t-BOC Protecting Group

To a suspension of the compound of Example 2 (1.86 g, 3.36 mmol) in MeOH (20 mL), was added 1.2 N of HCl in ethyl acetate (74 mL) slowly. The yellow cloudy mixture was stirred at room temperture under N₂. After 6.5 h, the reaction mixture was concentrated in vacuo to give a yellow solid. After purification by crystallization in (60:40) MeOH/IPA, 1.41 g, (80.1% yield, 96.9 area % by HPLC) of the hydrochloride salt of the titled compound was obtained as a light yellow solid. ¹H NMR (DMSO, ppm) δ 11.1 (bs, 1H), 9.98 (s, 1H), 9.36 (bs, 2H), 9.13 (s, 1H), 9.00 (s, 1H, 7.75 (dd, 1H, J=3 Hz, 6 Hz), 7.65 (s, 1H), 7.55 (t, 1H, J=8 Hz), 7.46-7.48 (m, 1H), 6.75-6.87 (m, 2H), 4.35 (q, 2H, J=5.1 Hz), 3.76-3.80 (m, 2H), 2.56 (t, 3H, J=4.0 Hz), 1.50 (t, 3H, J=5.3 Hz); ¹³C NMR (DMSO, ppm) δ 163.14, 155.59, 155.36, 153.59, 149.46, 135.77, 135.14, 129.6, 129.0, 128.3, 127.1, 120.3, 120.1, 117.8, 117.6, 116.9, 115.0, 112.9, 87.2, 65.7, 48.6, 32.4, 14.4; IR (Nujol): νₘₐₓ 3580, 3497, 3192, 2900, 2691, 2403, 2224, 1682, 1640, 1584, 1536, 1492, 1460, 1377, 1314, 1264, 1193, 885, 729 cm⁻¹.

### EXAMPLE 4

### (E)N-{4-[3-Chloro-4-(2-pyridinylmethoxy)anilino]-3-cyano-7-ethoxy-6-quinolinyl}-4-[N-(t-butyloxycarbonyl)methylamino]-2-butenamide

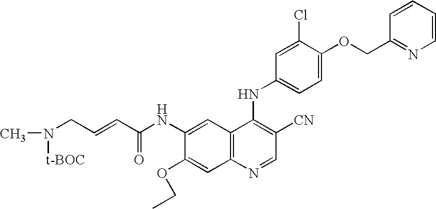

### Preparation of methyl 4-[N-(tert-butyloxycarbonyl)methylamino]crotonate

Cool a 4-neck 1-L flask to −15 to −25° C. Charge an addition funnel with methyl 4-bromocrotonate (25.0 g, 0.118 mole at 85% strength) in THF (125 ml). Charge a second addition funnel with 2.0 M methylamine solution in THF (149 ml, 0.30 mole, 2.5 eq). Add both solutions dropwise and simultaneously over 30 min maintaining the pot temperature at to −15 to −25° C. Warm the mixture to −10 to −5° C. and hold for 2 h. Add 2.0 M methylamine solution in THF (50 ml, 0.10 mole) if necessary to complete the reaction. When no starting material is detected by TLC (9:1 heptane:EtOAc), cool the reaction mixture to −60 to −65° C. Add triethylamine (60.0 g, 82.5 ml, 0.59 mole, 5 eq). Add dropwise a solution of di-t-butyl dicarbonate (103.75 g, 0.475 mole, 4 eq) in THF (250 ml) over 1 h. Hold the mixture overnight while allowing it to warm to room temperature. Check that the reaction is complete by TLC (9:1 heptane:EtOAc). The precipitates are filtered off and the mixture is concentrated to an oil. The oil is re-dissolved in CH₂Cl₂ (250 ml), washed consecutively with water (125 ml), 1 N HCl (125 ml) and water (2×125 ml). The organic layer is dried over sodium sulfate (50 g) for 10 mins. The mixture is filtered and concentrated to an oil, which was passed through a silica gel pad to obtain N-t-Boc-4-N-methylaminocrotonate (19.7 g, 73%). ¹H NMR (DMSO, ppm) δ 6.79 (dt, 1H, CH₂CH═CH), 5.81 (dt, 1H, CH₂CH═CH), 3.96 (m, 2H, NCH₂), 3.67 (s, 3H, OCH₃), 2.78 (s, 3H, NCH₃), 1.37 (s, 9H, t-butyl).

### Preparation of methyl 4-[N-(tert-butyloxycarbonyl)methylamino]crotonic acid

Charge a 3-neck 1-L flask with N-t-Boc-4-N-methylaminocrotonate (25.0 g, 0.11 mole) in THF (290 ml) and cool to 0-5° C. Dissolve a solution of potassium hydroxide (29.0 g, 85%, 0.44 mole, 4.0 eq) in water (145 ml) and add dropwise over 30 min keeping the pot temperature at 0-10° C. The pH at this stage is 11-12. Stir for 15 min, then warm the mixture to room temperature overnight. After reaction determined to be complete by TLC (8:2 heptane:EtOAc), the reaction mixture is concentrated under vacuo. The aqueous mixture is washed with CH₂Cl₂ (3×100 ml), and then acidified to pH 1-2 with 10% HCl (˜150 ml). The mixture is extracted with CH₂Cl₂ (3×150 ml). The combined organics are dried over Na₂SO₄, filtered and concentrated under vacuo to give N-t-Boc-4-N-methylaminocrotonic acid (18.0 g, 75%) as a light yellow oil. ¹H NMR (DMSO, ppm) δ 12.28 (bs, 1H, COOH, D₂O exchangeable), 6.75 (dt, 1H, CH₂CH═CH), 5.75 (dt, 1H, CH₂CH═CH), 3.96 (m, 2H, NCH₂), 2.78 (s, 3H, NCH₃), 1.39 (s, 9H, t-butyl).

### EXAMPLE 5

### Preparation of (E)N-{4-[3-Chloro-4-(2-pyridinylmethoxy)anilino]-3-cyano-7-ethoxy-6-quinolinyl)-4-[N-(t-butyloxycarbonyl)methylamino]-2-butenamide

A solution of N-t-Boc-4-N-methylaminocrotonic acid (18.0 g, 84 mmole, 2.0 eq) in CH₂Cl₂ (171 ml) was cooled to 0-10° C. Pyridine (68 ml, 840 mmol, 20 eq) and DCC (17.5 g, 84 mmole, 2 eq) were added. A suspension of 6-amino-N-4-[3-Chloro-4-(2-pyridinylmethoxy)anilino]-3-cyano-7-ethoxy-6-quinoline (19.0 g, 42 mmole) in CH₂Cl₂ (143 ml) was added dropwise and the reaction mixture was held for 5.5 days and monitored by HPLC. The reaction mixture was then diluted with CH₂Cl₂ (120 ml). The mixture was filtered and filtrates were washed with water (2×100 ml), 1N HCl (2×60 ml) and water (2×100 mL), dried over MgSO₄, filtered and concentrated under vacuo to give the crude product as a brown red residue. The crude product was purified by chromatography (CH₂Cl₂ with 0 to 2% MeOH) to give 15.0 g (56%, ˜57% strength by HPLC area) of (E)N-{4-[3-Chloro-4-(2-pyridinylmethoxy)anilino]-3-cyano-7-ethoxy-6-quinolinyl)-4-[N-(t-butyloxycarbonyl)methylamino]-2-butenamide. ¹H NMR (DMSO, ppm) δ 9.61 (s, 1H, NH), 9.57 (s, 1H, NH), 8.91 (s, 1H, Ar), 8.60 (m, 1H, Ar), 8.47 (s, 1H, Ar), 7.87 (m, 1H, Ar), 7.58 (m, 1H, Ar), 7.39-7.18 (m, 5H, Ar), 6.74 (dt, 1H, CH₂CH═CH), 6.47 (d, 1H, CH₂CH═CH), 5.29 (s, 2H, —OCH₂Pyr), 4.32 (q, 2H, OCH₂CH₃), 3.99 (m, 2H, NCH₂), 3.32 (s, 3H, NCH₃), 1.41 (s, 9H, (CH₃)₃C).

### EXAMPLE 6

### (E)N-{4-[3-Chloro-4-(2-pyridinylmethoxy)anilino]-3-cyano-7-ethoxy-6-quinolinyl}-4-(methylamino)-2-butenamide

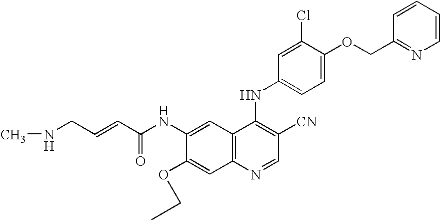

### Preparation of (E)N-{4-[3-Chloro-4-(2-pyridinylmethoxy)anilino]-3-cyano-7-ethoxy-6-quinolinyl}-4-(methylamino)-2-butenamide free base

To a suspension of (E)N-{4-[3-Chloro-4-(2-pyridinylmethoxy)anilino]-3-cyano-7-ethoxy-6-quinolinyl)-4-[N-(t-butyloxycarbonyl)methylamino]-2-butenamide (14.0 g, 21.8 mmole) in methanol (120 ml) was added 1.1 N HCl in ethyl acetate (515 ml) dropwise over 2 h. The resulting yellow suspension mixture was stirred at room temperature for 7.5 h. The reaction mixture was basified by adding 5% aq. NaOH (500 ml) to pH 8. The organic layer was separated, and aq. layer was extracted with EtOAc/MeOH (3×200 mi, 9:1), CH₂Cl₂/MeOH (3×200 ml, 9:1). The combined organic layers were dried over MgSO₄, and concentrated. The residue was purified by chromatography to produce 7.8 g, which was slurried in cold MeOH (210 ml) and filtered to obtain 5.23 g of the titled product (44%,) after washing with cold MeOH (65 ml) and vacuum drying at room temperature. ¹H NMR (CDCl₃, ppm) δ 9.17 (s, 1H, NH), 8.59 (m, 1H, NH), 8.41 (s, 1H, Ar), 7.97 (s, 1H, Ar), 7.92 (s, 1H, Ar), 7.76 (m, 1H, Ar), 7.64 (d, 1H, Ar), 7.24 (m, 1H, Ar), 7.14 (m, 2H, Ar), 7.09 (m, 1H, Ar), 6.89 (m, 1H, Ar), 6.83 (d, 1H, CH₂CH═CH), 6.14 (dt, 1H, CH₂CH═CH), 5.24 (s, 2H, —OCH₂Pyr), 4.20 (q, 2H, —OCH₂CH₃), 3.38 (m, 2H, NCH₂CH), 2.11 (s, 3H, NCH₃), 1.52 (t, 3H, CH₂CH₃). ¹³C NMR: δ (CDCl₃) 164.6, 156.8, 152.6, 151.2, 150.6, 149.3, 147.4, 145.5, 137.2, 133.0, 128.0, 127.6, 124.8, 123.7, 123.5, 123.0, 121.4, 117.3, 113.7, 113.3, 109.8, 108.8, 88.0, 71.6, 65.2, 52.5, 36.5, 14.8.

### EXAMPLE 7

### (E)N-{4-[3-Chloro-4-(2-pyridinylmethoxy)anilino]-3-cyano-7-ethoxy-6-quinolinyl)-4-(methylamino)-2-butenamide hydrochloride

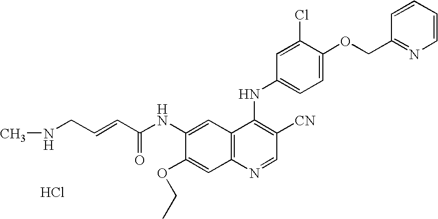

To a solution of (E)N-{4-[3-Chloro-4-(2-pyridinylmethoxy)anilino]-3-cyano-7-ethoxy-6-quinolinyl}-4-(methylamino)-2-butenamide free base (4.40 g, 8.12 mmole) dissolved in a mixture of methanol (10 ml) and methylene chloride (50 ml). The solution was added dropwise over 15-20 mins. to a solution of 1.28 N HCl in ethyl acetate (75 ml) and then rinsed with methylene chloride (5 ml). Some fuming occurs and precipitates formed. The mixture is held at room temperature for 20 mins., filtered and washed with ethyl acetate (20 ml). The yellow solids are dried under pump vacuum for 3 h at room temperature to provide 4.24 g (90% yield) of the titled compound. ¹H NMR (DMSO, ppm) δ 11.43 (bs, 1H, NH), 10.04 (s, 114, NH), 9.74 (bd, 2H, NH, HCl), 9.18 (s, 1H, Ar), 9.04 (s, 1H, Ar), 8.91 (d, 1H, Ar), 8.50 (dt, 1H, Ar), 8.05 (d, 1H, Ar), 7.93 (t, 1H, Ar), 7.85 (s, 1H, Ar), 7.71 (d, 1H, Ar), 7.49 (dd, 1H, Ar), 7.42 (d, I H, Ar), 6.97-6.85 (dt, I H, CH₂CH═CH), 6.81 (d, 1H, CH₂CH═CH), 5.66 (s, 2H, —OCH₂Pyr), 4.33 (q, 2H, —OCH₂CH₃), 3.78 (dd, 2H, NHCH₂CH), 2.54 (t, 3H, NCH₃), 1.51 (t, 3H, CH₂CH₃). ¹³C NMR: δ (DMSO-d₆) 14.7, 32.6, 48.9, 66.2, 68.2, 86.8, 101.6, 112.7, 114.6, 115.3, 117.9, 122.6, 125.5, 126.6, 127.7, 129.2, 129.5, 129.9, 132.5, 135.6, 137.7, 144.4, 145.1, 148.6, 152.4, 153.3, 154.8, 156.4, 163.6.

Analysis

NMR spectra were recorded on a GE QE 300, a Bruker DPX 301 or a Varian Inova 300 at 300 MHz (¹H) and at 75 or 300 MHz (¹³C) and chemical shifts were identified in ppm relative to TMS internal standard. Analytical and preparative TLCs were performed on Silica Gel 60 F-254 pre-coated plates obtained from EM Science. Compounds were visualized using UV at 254 nm, bromocresol green indicator, or phosphomolybdic acid reagents (20 wt % in EtOH). HPLC analysis was determined on a Waters Alliance 2695 HPLC instrument equipped with a PDA (Model 2996) detector. IR spectra were recorded on a Mattson 5020 FT-IR.

Biological Data

Kinase Assays

The activity of (E)N-{4-[3-chloro-4-(2-pyridinylmethoxy)anilino]-3-cyano-7-ethoxy-6-quinolinyl}-4-(methylamino)-2-butenamide, example 6, was first determined in a HER-2 autophosphorylation assay using the cytoplasmic kinase domain of HER-2. (E)N-{4-[3-chloro-4-(2-pyridinylmethoxy)anilino]-3-cyano-7-ethoxy-6-quinolinyl}-4-(methylamino)-2-butenamide reduced receptor autophosphorylation by 50% (IC₅₀) at 3.4 nM (Table 1). This is similar to the IC₅₀ of the dimethylamino compound from which it is metabolized from, (E)N-{4-[3-Chloro-4-(2-pyridinylmethoxy)anilino]-3-cyano-7-ethoxy-6-quinolinyl}-4-(dimethylamino)-2-butenamide, in this assay (1.1 nM).

TABLE 1
HER-2 AUTOPHOSPHORYLATION ASSAY

| COMPOUND | IC₅₀ (nM)^{a} |
|---|---|
| Dimethylamino | 1.1 ± 0.6 (2) |
| Des-methylamino, (Ex. 6) | 3.4 ± 1.6 (2) |

Purified recombinant C-terminal fragment of HER-2 was incubated with ATP in the absence or presence of a range of compound concentrations.
Autophosphorylation of the receptor was determined using phosphotyrosine antibodies. The concentration of compound that inhibits phosphorylation by 50% is shown.
^{a}Mean and SE with the number of independent determinations in parentheses.

The activity of (E)N-{4-[3-chloro-4-(2-pyridinylmethoxy)anilino]-3-cyano-7-ethoxy-6-quinolinyl}-4-(methylamino)-2-butenamide was also tested in a proprietary kinase assay (³³PanQinase® assay; ProQinase, Freiburg, Germany) using HER-2 and the related ErbB kinases, EGFR and HER-4, and is shown in Table 2. Here, the ability of the compound to inhibit phosphorylation of a substrate peptide [poly(Glu-Tyr) or poly(Ala-Glu-Lys-Tyr)] was determined. (E)N-{4-[3-chloro-4-(2-pyridinylmethoxy)anilino]-3-cyano-7-ethoxy-6-quinolinyl}-4-(methylamino)-2-butenamide inhibited the activity of HER-2 (IC₅₀=21 nM), EGFR (IC₅₀=7 nM) and HER-4 (IC₅₀=13 nM). When compared to the dimethylamino derivative, (E)N-{4-[3-chloro-4-(2-pyridinylmethoxy)anilino]-3-cyano-7-ethoxy-6-quinolinyl}-4-(methylamino)-2-butenamide showed similar activity against all three kinases in this assay.

TABLE 2
ErbB SUBSTRATE PHOSPHORYLATION ASSAY

| | IC₅₀ (nM) | | |
|---|---|---|---|
| COMPOUND | EGFR | HER-2 | HER-4 |
| Dimethylamino | 12 | 39 | 19 |
| Des-methylamino, | 7 | 21 | 13 |
| (Ex. 6) | | | |

Purified recombinant C-terminal domain of the ErbB receptors were incubated with [γ³³P]-ATP and peptide substrate in the absence or presence of a range of compound concentrations. The concentration of compound that inhibits phosphorylation of the peptide by 50% is shown.

Cell Proliferation Assays

(E)N-{4-[3-chloro-4-(2-pyridinylmethoxy)anilino]-3-cyano-7-ethoxy-6-quinolinyl}-4-(methylamino)-2-butenamide inhibited the proliferation of 3T3/neu, a mouse fibroblast cell line transfected with the HER-2 oncogene (IC₅₀=40 nM), while having a minimal effect on the isogenic untransfected cells (IC₅₀=5460 nM; Table 3). It also inhibited two other breast cancer cell lines that overexpress HER-2, BT474 and SK-Br-3 (IC₅₀ 3, 34 nM, respectively). Consistent with the effect in the EGFR kinase assay, the des-methylamino compound also inhibited the EGFR-dependent cell line, A431 (IC₅₀=67 nM). It was substantially less active against SW620 colon carcinoma cells that express neither receptor (IC₅₀=1604 nM). When compared with the dimethylamino compound, (E)N-{4-[3-chloro-4-(2-pyridinylmethoxy)anilino]-3-cyano-7-ethoxy-6-quinolinyl}-4-(methylamino)-2-butenamide was 8-fold less potent against 3T3/neu cells, but as potent against other HER-2 and EGFR-expressing cell lines.

TABLE 3
CELL PROLIFERATION ASSAYS

| | | | IC₅₀ (nM)^{a} | |
|---|---|---|---|---|
| | | | | Des- |
| | | | | myhtlamino |
| CELL LINE | HER-2 | EGFR | Dimethylamino | (Ex. 6) |
| 3T3 | − | − | 2191 ± 700 (2) | 5460 ± 659 (3) |
| 3T3/neu | +++ | − | 5 ± 0.2 (2) | 40 ± 4 (3) |
| A431 | + | +++ | 64 ± 3 (2) | 67 ± 7 (3) |
| SK-Br-3 | +++ | − | 36 ± 20 (2) | 34 ± 4 (3) |
| BT474 | +++ | + | 2 ± 0.2 (2) | 3 ± 0.5 (3) |
| SW620 | − | − | 1215 ± 246 (2) | 1604 ± 138 (3) |

Cells were incubated with various concentrations of compound for 2 days (6 days for BT474). Cell survival was determined using a protein binding dye assay (SRB). The concentration of compound that reduces cell survival by 50% is shown. Relative expression of EGFR and HER-2 in the cell lines are indicated.
^{a}Mean and SE are shown with the number of independent determinations in parentheses.

In Vivo Activity

The in vivo activity of (E)N-{4-[3-chloro-4-(2-pyridinylmethoxy)anilino]-3-cyano-7-ethoxy-6-quinolinyl}-4-(methylamino)-2-butenamide was first studied in xenografts of 3T3/neu. Daily oral administration of the compound between 5 and 40 mg/kg/day had no effect on tumor growth, while good antitumor activity was observed for dimethylamino compound at 40 mg/kg/day (74% inhibition, day 18; data not shown). In a second independent test, increasing the dose of the des-methylamino compound to 100 mg/kg/day also had no effect on tumor growth (Table 4), while the dimethylamino compound showed good antitumor activity between 40 and 80 mg/kg/day (93-100% inhibition, day 14).

In animals bearing BT474 xenografts, treatment of animals with (E)N-{4-[3-chloro-4-(2-pyridinylmethoxy)anilino]-3-cyano-7-ethoxy-6-quinolinyl}-4-(methylamino)-2-butenamide had no effect at 10-40 mg/kg/day, as shown in Table 4. In contrast, treatment with the dimethylamino compound at the same doses showed good antitumor activity. Maximum tumor inhibition was observed on day 21 ranging from 64% (10 mg/kg/day) to 97% (40 mg/kg/day).

TABLE 4
EFFECT ON TUMOR XENOGRAFTS

| | | % T/C (p-value) | | | | |
|---|---|---|---|---|---|---|
| Compound | Dose | Day 7/8* | Day 14 | Day 21 | Day 28 | Day 35 |
| 3T3/neu tumors | | | | | | |
| Des-methylamino, (Ex. 6) | 100 | 140 (1.00) | 113 (0.93) | | | |
| Dimethylamino | 80 | 0 (<0.01) | 0 (<0.01) | | | |
| | 40 | 1 (<0.01) | 7 (<0.01) | | | |
| BT474 tumors | | | | | | |
| Des-methylamino, (Ex. 6) | 40 | 91 (0.49) | 75 (0.26) | 75 (0.16) | 89 (0.26) | 89 (0.37) |
| | 10 | 71 (0.13) | 75 (0.30) | 85 (0.43) | 86 (0.43) | 108 (0.74) |
| Dimethylamino | 40 | 29 (<0.01) | 9 (<0.01) | 3 (<0.01) | 6 (<0.01) | 11 (<0.01) |
| | 10 | 50 (<0.01) | 44 (0.01) | 36 (<0.01) | 47 (0.01) | 72 (0.22) |
| Mice were implanted with 2 × 10⁶ 3T3/neu cells or a single BT474 tumor fragment (˜3 mm³). Animals bearing 3T3/neu xenografts were treated with vehicle or compound (10 mice per group) on days 1-10 (PO), beginning the day after implantation. | | | | | | |
| # For BT474, tumors were staged when they reached a size of 65-100 mg, animals were assigned to treatment groups (5 mice per group, 10 for vehicle controls), and treated with vehicle or compounds once-daily (PO) on days 1-20. | | | | | | |

Tumor mass ([length × width²]/2) was determined were once per week. For BT474 xenografts, tumor growth was expressed as Relative Tumor Growth: the ratio of mean tumor mass to the tumor mass on the staging day.
% T/C: ratio of tumor burden in treated animals to control animals, expressed as a percentage. Statistical signficance was determined using the Students t-test;
p < 0.05 is considered signficant. Doses shown are in mg/kg/day.
*day 8 data for 3T3/neu xenografts; day 7 data for BT474 xenografts.

We claim:

1. A method of preparing a compound of formula (I): 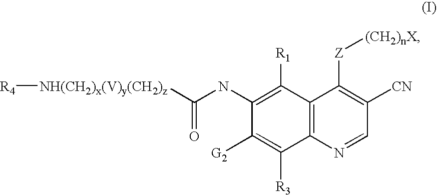
comprising the step of deprotecting a compound of formula (II): 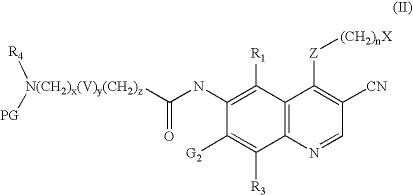
wherein:
X is cycloalkyl of 3 to 7 carbon atoms, which may be optionally substituted with one or more alkyl of 1 to 6 carbon atom groups; or is a pyridinyl, pyrimidinyl, or phenyl ring; wherein the pyridinyl, pyrimidinyl, or phenyl ring may be optionally mono- di-, or tri-substituted with a substituent selected from the group consisting of halogen, alkyl of 1-6 carbon atoms, alkenyl of 2-6 carbon atoms, alkynyl of 2-6 carbon atoms, azido, hydroxyalkyl of 1-6 carbon atoms, halomethyl, alkoxymethyl of 2-7 carbon atoms, alkanoyloxymethyl of 2-7 carbon atoms, alkoxy of 1-6 carbon atoms, alkylthio of 1-6 carbon atoms, hydroxy, trifluoromethyl, cyano, nitro, carboxy, carboalkoxy of 2-7 carbon atoms, carboalkyl of 2-7 carbon atoms, phenoxy, phenyl, thiophenoxy, benzoyl, benzyl, amino, alkylamino of 1-6 carbon atoms, dialkylamino of 2 to 12 carbon atoms, phenylamino, benzylamino, alkanoylamino of 1-6 carbon atoms, alkenoylamino of 3-8 carbon atoms, alkynoylamino of 3-8 carbon atoms, carboxyalkyl of 2-7 carbon atoms, carboalkoxyalkyl of 3-8 carbon atoms, aminoalkyl of 1-5 carbon atoms, N-alkylaminoalkyl of 2-9 carbon atoms, N,N-dialkylaminoalkyl of 3-10 carbon atoms, N-alkylaminoalkoxy of 2-9 carbon atoms, N,N-dialkylaminoalkoxy of 3-10 carbon atoms, mercapto, and benzoylamino; or
X is a bicyclic aryl or bicyclic heteroaryl ring system of 8 to 12 atoms where the bicyclic heteroaryl ring contains 1 to 4 heteroatoms selected from N, O, and S with the proviso that the bicyclic heteroaryl ring does not contain O—O, S—S, or S—O bonds and where the bicyclic aryl or bicyclic heteroaryl ring may be optionally mono- di-, tri, or tetra-substituted with a substituent selected from the group consisting of halogen, oxo, thio, alkyl of 1-6 carbon atoms, alkenyl of 2-6 carbon atoms, alkynyl of 2-6 carbon atoms, azido, hydroxyalkyl of 1-6 carbon atoms, halomethyl, alkoxymethyl of 2-7 carbon atoms, alkanoyloxymethyl of 2-7 carbon atoms, alkoxy of 1-6 carbon atoms, alkylthio of 1-6 carbon atoms, hydroxy, trifluoromethyl, cyano, nitro, carboxy, carboalkoxy of 2-7 carbon atoms, carboalkyl of 2-7 carbon atoms, phenoxy, phenyl, thiophenoxy, benzoyl, benzyl, amino, alkylamino of 1-6 carbon atoms, dialkyl amino of 2 to 12 carbon atoms, phenylamino, benzylamino, alkanoylamino of 1-6 carbon atoms, alkenoylamino of 3-8 carbon atoms, alkynoylamino of 3-8 carbon atoms, carboxyalkyl of 2-7 carbon atoms, carboalkoxyalkyl of 3-8 carbon atoms, aminoalkyl of 1-5 carbon atoms, N-alkylaminoalkyl of 2-9 carbon atoms, N,N-dialkylaminoalkyl of 3-10 carbon atoms, N-alkylaminoalkoxy of 2-9 carbon atoms, N,N-dialkylaminoalkoxy of 3-10 carbon atoms, mercapto, and benzoylamino; or
X is a radical having the formula: 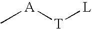
wherein
A is a pyridinyl, pyrimidinyl, or phenyl ring; wherein the pyridinyl, pyrimidinyl, or phenyl ring may be optionally mono- or di-substituted with a substituent selected from the group consisting of halogen, alkyl of 1-6 carbon atoms, alkenyl of 2-6 carbon atoms, alkynyl of 2-6 carbon atoms, azido, hydroxyalkyl of 1-6 carbon atoms, halomethyl, alkoxymethyl of 2-7 carbon atoms, alkanoyloxymethyl of 2-7 carbon atoms, alkoxy of 1-6 carbon atoms, alkylthio of 1-6 carbon atoms, hydroxy, trifluoromethyl, cyano, nitro, carboxy, carboalkoxy of 2-7 carbon atoms, carboalkyl of 2-7 carbon atoms, phenoxy, phenyl, thiophenoxy, benzoyl, benzyl, amino, alkylamino of 1-6 carbon atoms, dialkylamino of 2 to 12 carbon atoms, phenylamino, benzylamino, alkanoylamino of 1-6 carbon atoms, alkenoylamino of 3-8 carbon atoms, alkynoylamino of 3-8 carbon atoms, carboxyalkyl of 2-7 carbon atoms, carboalkoxyalkyl of 3-8 carbon atoms, aminoalkyl of 1-5 carbon atoms, N-alkylaminoalkyl of 2-9 carbon atoms, N,N-dialkylaminoalkyl of 3-10 carbon atoms, N-alkylaminoalkoxy of 2-9 carbon atoms, N,N-dialkylaminoalkoxy of 3-10 carbon atoms, mercapto, and benzoylamino;
T is bonded to a carbon of A and is:
—NH(CH₂)ₘ—, —O(CH₂)ₘ—, —S(CH₂)ₘ—, —NR(CH₂)ₘ—, —(CH₂)ₘ—, —(CH₂)ₘNH—, —CH₂)ₘO—, —(CH₂)ₘS—, or —(CH₂)ₘNR—;
L is an unsubsitituted phenyl ring or a phenyl ring mono-, di-, or tri-substituted with a substituent selected from the group consisting of halogen, alkyl of 1-6 carbon atoms, alkenyl of 2-6 carbon atoms, alkynyl of 2-6 carbon atoms, azido, hydroxyalkyl of 1-6 carbon atoms, halomethyl, alkoxymethyl of 2-7 carbon atoms, alkanoyloxymethyl of 2-7 carbon atoms, alkoxy of 1-6 carbon atoms, alkylthio of 1-6 carbon atoms, hydroxy, trifluoromethyl, cyano, nitro, carboxy, carboalkoxy of 2-7 carbon atoms, carboalkyl of 2-7 carbon atoms, phenoxy, phenyl, thiophenoxy, benzoyl, benzyl, amino, alkylamino of 1-6 carbon atoms, dialkylamino of 2 to 12 carbon atoms, phenylamino, benzylamino, alkanoylamino of 1-6 carbon atoms, alkenoylamino of 3-8 carbon atoms, alkynoylamino of 3-8 carbon atoms, carboxyalkyl of 2-7 carbon atoms, carboalkoxyalkyl of 3-8 carbon atoms, aminoalkyl of 1-5 carbon atoms, N-alkylaminoalkyl of 2-9 carbon atoms, N,N-dialkylaminoalkyl of 3-10 carbon atoms, N-alkylaminoalkoxy of 2-9 carbon atoms, N,N-dialkylaminoalkoxy of 3-10 carbon atoms, mercapto, and benzoylamino; or
L is a 5- or 6-membered heteroaryl ring where the heteroaryl ring contains 1 to 3 heteroatoms selected from N, O, and S, with the proviso that the heteroaryl ring does not contain O—O, S—S, or S—O bonds, and where the heteroaryl ring is optionally mono- or di-substituted with a substituent selected from the group consisting of halogen, oxo, thio, alkyl of 1-6 carbon atoms, alkenyl of 2-6 carbon atoms, alkynyl of 2-6 carbon atoms, azido, hydroxyalkyl of 1-6 carbon atoms, halomethyl, alkoxymethyl of 2-7 carbon atoms, alkanoyloxymethyl of 2-7 carbon atoms, alkoxy of 1-6 carbon atoms, alkylthio of 1-6 carbon atoms, hydroxy, trifluoromethyl, cyano, nitro, carboxy, carboalkoxy of 2-7 carbon atoms, carboalkyl of 2-7 carbon atoms, phenoxy, phenyl, thiophenoxy, benzoyl, benzyl, amino, alkylamino of 1-6 carbon atoms, dialkylamino of 2 to 12 carbon atoms, phenylamino, benzylamino, alkanoylamino of 1-6 carbon atoms, alkenoylamino of 3-8 carbon atoms, alkynoylamino of 3-8 carbon atoms, carboxyalkyl of 2-7 carbon atoms, carboalkoxyalkyl of 3-8 carbon atoms, aminoalkyl of 1-5 carbon atoms, N-alkylaminoalkyl of 2-9 carbon atoms, N,N-dialkylaminoalkyl of 3-10 carbon atoms, N-alkylaminoalkoxy of 2-9 carbon atoms, N,N-dialkylaminoalkoxy of 3-10 carbon atoms, mercapto, and benzoylamino;
V is ethylene or acetylene;
PG is an amine protecting group;
R₄ is alkyl of 1 to 6 carbons;
Z is NR^{z′}, O or S, wherein R^{z′} is H or C₁-C₆ alkyl;
R₁, G₂, and R₃ are each, independently, hydrogen, halogen, alkyl of 1-6 carbon atoms, alkenyl of 2-6 carbon atoms, alkynyl of 2-6 carbon atoms, alkenyloxy of 2-6 carbon atoms, alkynyloxy of 2-6 carbon atoms, hydroxymethyl, halomethyl, alkanoyloxy of 1-6 carbon atoms, alkenoyloxy of 3-8 carbon atoms, alkynoyloxy of 3-8 carbon atoms, alkanoyloxymethyl of 2-7 carbon atoms, alkenoyloxymethyl of 4-9 carbon atoms, alkynoyloxymethyl of 4-9 carbon atoms, alkoxymethyl of 2-7 carbon atoms, alkoxy of 1-6 carbon atoms, alkylthio of 1-6 carbon atoms, alkylsulphinyl of 1-6 carbon atoms, alkylsulphonyl of 1-6 carbon atoms, alkylsulfonamido of 1-6 carbon atoms, alkenylsulfonamido of 2-6 carbon atoms, alkynylsulfonamido of 2-6 carbon atoms, hydroxy, trifluoromethyl, trifluoromethoxy, cyano, nitro, carboxy, carboalkoxy of 2-7 carbon atoms, carboalkyl of 2-7 carbon atoms, phenoxy, phthalimide, phenyl, thiophenoxy, benzyl, amino, hydroxyamino, alkoxyamino of 1-4 carbon atoms, alkylamino of 1-6 carbon atoms, dialkylamino of 2 to 12 carbon atoms, N-alkylcarbamoyl, N,N-dialkylcarbamoyl, N-alkyl-N-alkenylamino of 4 to 12 carbon atoms, N,N-dialkenylamino of 6-12 carbon atoms, phenylamino, benzylamino, 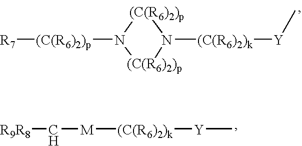
R₇—(C(R₆)₂)_{g}—Y—, R₇—(C(R₆)₂)ₚ-M-(C(R₆)₂)ₖ—Y—, or Het-(C(R₆)₂)_{q}W—(C(R₆)₂—Y—;
or R₁ and R₃ are as defined above and G₂ is R₂—NH—;
or if any of the substituents R₁, G₂ or R₃ are located on contiguous carbon atoms then they may be taken together as the divalent radical —O—C(R₆)₂—O—;
Y is a divalent radical selected from the group consisting of 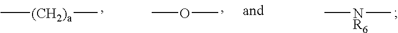
R₇ is —NR₆R₆, —OR₆, -J, —N(R₆)₃⁺, or —NR₆(OR₆);
M is >NR₆, —O—, >N—(C(R₆)₂)ₚNR₆R₆, or >N—(C(R₆)₂)ₚ—OR₆;
W is >NR₆, —O— or is a bond;
Het is is selected from the group consisting of morpholine, thiomorpholine, thiomorpholine S-oxide, thiomorpholine S,S-dioxide, piperidine, pyrrolidine, aziridine, pyridine, imidazole, 1,2,3-triazole, 1,2,4-triazole, thiazole, thiazolidine, tetrazole, piperazine, furan, thiophene, tetrahydrothiophene, tetrahydrofuran, dioxane, 1,3-dioxolane, tetrahydropyran, and 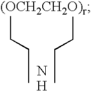
wherein Het is optionally mono- or di-substituted on carbon or nitrogen with R₆, optionally mono- or di-substituted on carbon with hydroxy, —N(R₆)₂, or —OR₆, optionally mono or di-substituted on carbon with the mono-valent radicals —C(R₆)₂)ₛOR₆ or —(C(R₆)₂)ₛN(R₆)₂, and optionally mono or di-substituted on a saturated carbon with divalent radicals —O— or —O(C(R₆)₂)ₛO—;
R₆ is hydrogen, alkyl of 1-6 carbon atoms, alkenyl of 2-6 carbon atoms, alkynyl of 2-6 carbon atoms, cycloalkyl of 1-6 carbon atoms, carboalkyl of 2-7 carbon atoms, carboxyalkyl (2-7 carbon atoms), phenyl, or phenyl optionally substituted with one or more halogen, alkoxy of 1-6 carbon atoms, trifluoromethyl, amino, alkylamino of 1-3 carbon atoms, dialkylamino of 2-6 carbon atoms, nitro, cyano, azido, halomethyl, alkoxymethyl of 2-7 carbon atoms, alkanoyloxymethyl of 2-7 carbon atoms, alkylthio of 1-6 carbon atoms, hydroxy, carboxyl, carboalkoxy of 2-7 carbon atoms, phenoxy, phenyl, thiophenoxy, benzoyl, benzyl, phenylamino, benzylamino, alkanoylamino of 1-6 carbon atoms, or alkyl of 1-6 carbon atoms; with the proviso that the alkenyl or alkynyl moiety is bound to a nitrogen or oxygen atom through a saturated carbon atom;
R₂, is selected from the group consisting of 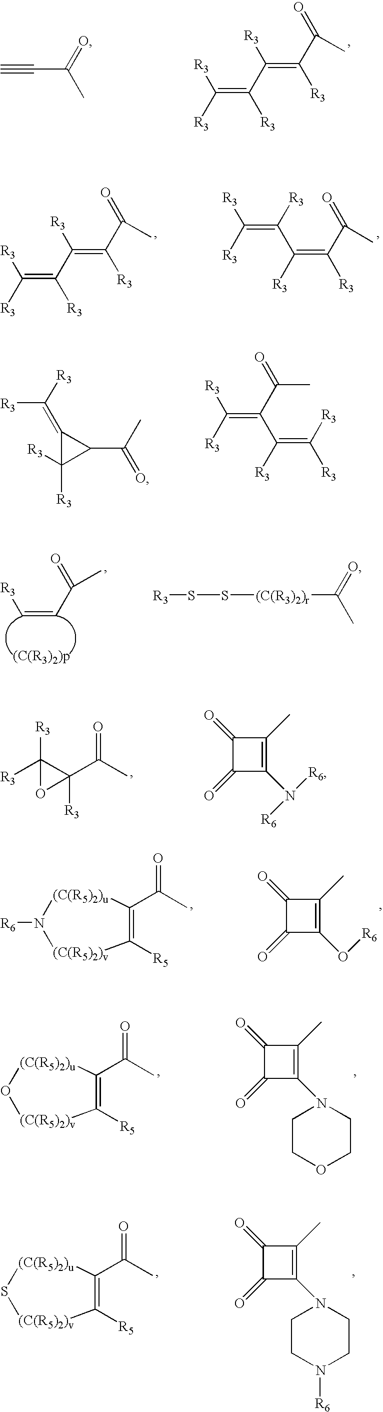 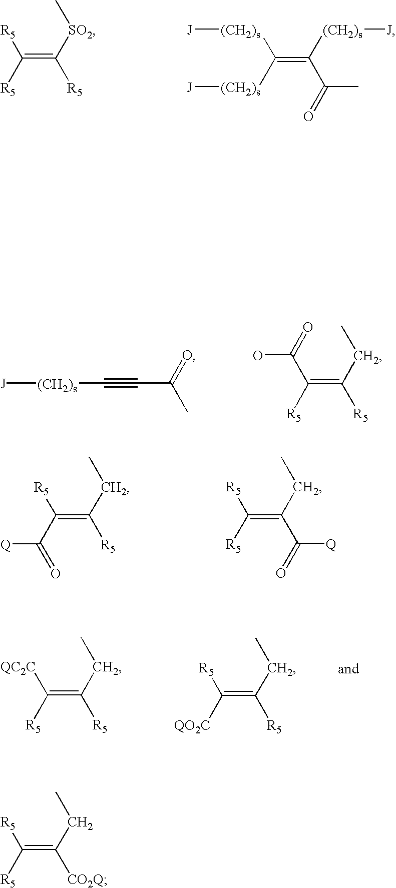
R₃ is independently hydrogen, alkyl of 1-6 carbon atoms, carboxy, carboalkoxy of 1-6 carbon atoms, phenyl, carboalkyl of 2-7 carbon atoms, 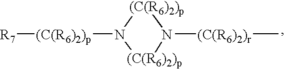
R₇—(C(R₆)₂)ₛ—,
R₇—(C(R₆)₂)ₚ-M-(C(R₆)₂)ᵣ—, R₈R₉—CH—
M-(C(R₆)₂)ᵣ—, or
Het-(C(R₆)₂)_{q}—W—(C(R₆)₂)ᵣ—;
R₅ is independently hydrogen, alkyl of 1-6 carbon atoms, carboxy, carboalkoxy of 1-6 carbon atoms, phenyl carboalkyl of 2-7 carbon atoms, 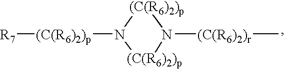
R₇—(C(R₆)₂)ₛ—,
R₇—(C(R₆)₂)ₚ-M-(C(R₆)₂)ᵣ—, R₈R₉—CH—
M-(C(R₆)₂)ᵣ—, or
Het-(C(R₆)₂)_{q}—W—(C(R₆)₂)ᵣ—;
R₈, and R₉ are each independently —(C(R₆)₂)ᵣNR₆R₆, or —(C(R₆)₂)ᵣOR₆;
J is independently hydrogen, chlorine, fluorine, or bromine;
Q is aklyl of 1-6 carbon atoms or hydrogen;
a=0 or 1;
g=1-6;
k=0-4;
n is 0-1;
m is 0-3;
p=2-4;
q=0-4;
r=1-4;
s=1-6;
u=0-4 and v=0-4, wherein the sum of u+v is 2-4;
x=0-3;
y=0-1; and
z=0-3;
or a pharmaceutically acceptable salt thereof.

2. The method of claim 1, further comprising the following provisos:
when R₆ is alkenyl of 2-7 carbon atoms or alkynyl of 2-7 carbon atoms, such alkenyl or alkynyl moiety is bound to a nitrogen or oxygen atom through a saturated carbon atom;
when Y is —NR₆— and R₇ is —NR₆R₆, —N(R₆)₃.N(R₈)₃⁺, or —NR₆(OR₆), then g=2-6;
when M is —O— and R₇ is —OR₆ then p=1-4;
when Y is —NR₆— then k=2-4;
when Y is —O— and M or W is —O— then k=1-4;
when W is not a bond with Het bonded through a nitrogen atom then q=2-4;
when W is a bond with Het bonded through a nitrogen atom and Y is —O— or —NR₆— then k=2-4; and
provided that L can be an unsubstituted phenyl ring only when m>0 and T is not —CH₂NH— or —CH₂O—.

3. The method of claim 1, further comprising the step of coupling an anilinoquinoline of formula (III): 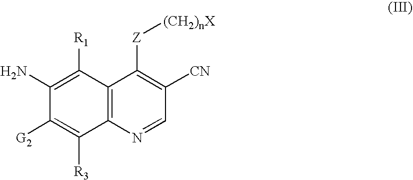
with an acid of formula (VII): 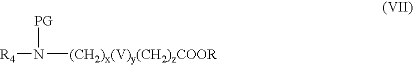
to form a compound of formula (II), wherein R is H, R₁, R₃, R₄, Z, G₂, V, PG, x, y, z, and n are as previously defined.

4. The method of claim 3, further comprising the steps of:
a. arylating a compound of formula (V): 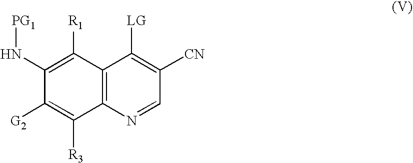
with a compound of formula HZ-(CH₂)ₙX to form an intermediate of formula (IV): 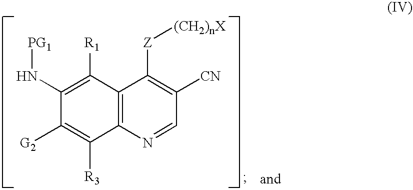
b. deprotecting the intermediate of formula (IV) to obtain the anilinoquinoline compound of formula (III);
wherein LG is selected from the group of halo, mesylate, tosylate and trifylate, PG₁ is an amine protecting group, and wherein R₁, R₃, R₄, Z, G₂, PG and n are as previously defined.

5. The method of claim 1, wherein PG is removed using an acid.

6. A method of preparing an acid of formula (VII): 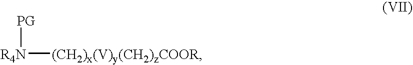
wherein:
R is H;
R₄ is H or alkyl of 1 to 6 carbon atoms;
V is ethylene or acetylene;
x is 1, 2 or 3;
y is 0 or 1;
z is 1, 2 or 3; and
PG is an amine protecting group;
comprising the step of hydrolysizing a corresponding ester of formula (VII′): 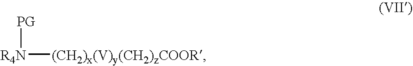
wherein R′ is alkyl of 1 to 6 carbon atoms or aryl, to form an acid of formula (VII).

7. The method of claim 6, further comprising the steps of:
a. alkylating a compound of formula (VI):
LG′-(CH₂)ₓ(V)_{y}(CH₂)_{z}COOR′ (VI)
with a primary amine of the formula NH₂R₄ to give an aminoester intermediate; and
b. subsequently protecting the aminoester intermediate by alkyating on a protecting group to form an ester of formula (VII′): 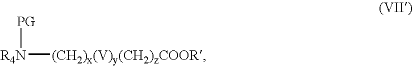
wherein LG′ is selected from halo, mesylate, tosylate and triflate, R′ is alkyl or aryl and R₄, V, PG, x, y, and z are as previously defined.

8. The method of claim 7, wherein PG is selected from the group consisting of t-BOC, acetyl, CH₃OC(O)—, EtOC(O)—, PhC(O)—, PhCH₂OC(O)—, Fmoc, Troc, Phenoc, and Teoc.

9. The method of claim 8, wherein the PG is t-BOC.

10. The method of claim 7, wherein (t-BOC)₂O is used in step b. to form the protecting group.

11. The method of claim 7, wherein z is 0, y is 1 and V is ethylene.

12. The method of claim 7, wherein R₄ is methyl, ethyl, propyl or isopropyl.

13. The method of claim 7, wherein the acid of formula (VII) is:

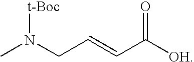

14. A method of preparing a compound of formula (I): 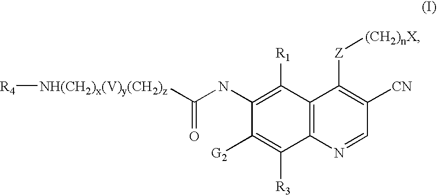
comprising the step of coupling an anilinoquinoline of formula (III): 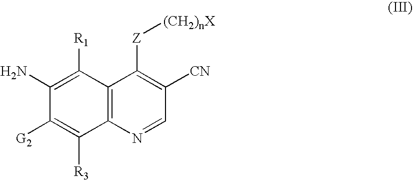
with an acid of formula (VII″):
R₄—NH.(CH₂)ₓ(V)_{y}(CH₂)_{z}COOR″ (VII″)
wherein:
R″ is H;
X is cycloalkyl of 3 to 7 carbon atoms, which may be optionally substituted with one or more alkyl of 1 to 6 carbon atom groups; or is a pyridinyl, pyrimidinyl, or phenyl ring; wherein the pyridinyl, pyrimidinyl, or phenyl ring may be optionally mono- di-, or tri-substituted with a substituent selected from the group consisting of halogen, alkyl of 1-6 carbon atoms, alkenyl of 2-6 carbon atoms, alkynyl of 2-6 carbon atoms, azido, hydroxyalkyl of 1-6 carbon atoms, halomethyl, alkoxymethyl of 2-7 carbon atoms, alkanoyloxymethyl of 2-7 carbon atoms, alkoxy of 1-6 carbon atoms, alkylthio of 1-6 carbon atoms, hydroxy, trifluoromethyl, cyano, nitro, carboxy, carboalkoxy of 2-7 carbon atoms, carboalkyl of 2-7 carbon atoms, phenoxy, phenyl, thiophenoxy, benzoyl, benzyl, amino, alkylamino of 1-6 carbon atoms, dialkylamino of 2 to 12 carbon atoms, phenylamino, benzylamino, alkanoylamino of 1-6 carbon atoms, alkenoylamino of 3-8 carbon atoms, alkynoylamino of 3-8 carbon atoms, carboxyalkyl of 2-7 carbon atoms, carboalkoxyalkyl of 3-8 carbon atoms, aminoalkyl of 1-5 carbon atoms, N-alkylaminoalkyl of 2-9 carbon atoms, N,N-dialkylaminoalkyl of 3-10 carbon atoms, N-alkylaminoalkoxy of 2-9 carbon atoms, N,N-dialkylaminoalkoxy of 3-10 carbon atoms, mercapto, and benzoylamino; or
X is a bicyclic aryl or bicyclic heteroaryl ring system of 8 to 12 atoms where the bicyclic heteroaryl ring contains 1 to 4 heteroatoms selected from N, O, and S with the proviso that the bicyclic heteroaryl ring does not contain O—O, S—S, or S—O bonds and where the bicyclic aryl or bicyclic heteroaryl ring may be optionally mono- di-, tri, or tetra-substituted with a substituent selected from the group consisting of halogen, oxo, thio, alkyl of 1-6 carbon atoms, alkenyl of 2-6 carbon atoms, alkynyl of 2-6 carbon atoms, azido, hydroxyalkyl of 1-6 carbon atoms, halomethyl, alkoxymethyl of 2-7 carbon atoms, alkanoyloxymethyl of 2-7 carbon atoms, alkoxy of 1-6 carbon atoms, alkylthio of 1-6 carbon atoms, hydroxy, trifluoromethyl, cyano, nitro, carboxy, carboalkoxy of 2-7 carbon atoms, carboalkyl of 2-7 carbon atoms, phenoxy, phenyl, thiophenoxy, benzoyl, benzyl, amino, alkylamino of 1-6 carbon atoms, dialkyl amino of 2 to 12 carbon atoms, phenylamino, benzylamino, alkanoylamino of 1-6 carbon atoms, alkenoylamino of 3-8 carbon atoms, alkynoylamino of 3-8 carbon atoms, carboxyalkyl of 2-7 carbon atoms, carboalkoxyalkyl of 3-8 carbon atoms, aminoalkyl of 1-5 carbon atoms, N-alkylaminoalkyl of 2-9 carbon atoms, N,N-dialkylaminoalkyl of 3-10 carbon atoms, N-alkylaminoalkoxy of 2-9 carbon atoms, N,N-dialkylaminoalkoxy of 3-10 carbon atoms, mercapto, and benzoylamino; or
X is a radical having the formula: 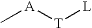
wherein
A is a pyridinyl, pyrimidinyl, or phenyl ring; wherein the pyridinyl, pyrimidinyl, or phenyl ring may be optionally mono- or di-substituted with a substituent selected from the group consisting of halogen, alkyl of 1-6 carbon atoms, alkenyl of 2-6 carbon atoms, alkynyl of 2-6 carbon atoms, azido, hydroxyalkyl of 1-6 carbon atoms, halomethyl, alkoxymethyl of 2-7 carbon atoms, alkanoyloxymethyl of 2-7 carbon atoms, alkoxy of 1-6 carbon atoms, alkylthio of 1-6 carbon atoms, hydroxy, trifluoromethyl, cyano, nitro, carboxy, carboalkoxy of 2-7 carbon atoms, carboalkyl of 2-7 carbon atoms, phenoxy, phenyl, thiophenoxy, benzoyl, benzyl, amino, alkylamino of 1-6 carbon atoms, dialkylamino of 2 to 12 carbon atoms, phenylamino, benzylamino, alkanoylamino of 1-6 carbon atoms, alkenoylamino of 3-8 carbon atoms, alkynoylamino of 3-8 carbon atoms, carboxyalkyl of 2-7 carbon atoms, carboalkoxyalkyl of 3-8 carbon atoms, aminoalkyl of 1-5 carbon atoms, N-alkylaminoalkyl of 2-9 carbon atoms, N,N-dialkylaminoalkyl of 3-10 carbon atoms, N-alkylaminoalkoxy of 2-9 carbon atoms, N,N-dialkylaminoalkoxy of 3-10 carbon atoms, mercapto, and benzoylamino;
T is bonded to a carbon of A and is:
—NH(CH₂)ₘ—, —O(CH₂)ₘ—, —S(CH₂)ₘ—, —NR(CH₂)ₘ—, —(CH₂)ₘ—, —(CH₂)ₘNH—, —(CH₂)ₘO—, —(CH₂)ₘS—, or —(CH₂)ₘNR—;
L is an unsubsitituted phenyl ring or a phenyl ring mono-, di-, or tri-substituted with a substituent selected from the group consisting of halogen, alkyl of 1-6 carbon atoms, alkenyl of 2-6 carbon atoms, alkynyl of 2-6 carbon atoms, azido, hydroxyalkyl of 1-6 carbon atoms, halomethyl, alkoxymethyl of 2-7 carbon atoms, alkanoyloxymethyl of 2-7 carbon atoms, alkoxy of 1-6 carbon atoms, alkylthio of 1-6 carbon atoms, hydroxy, trifluoromethyl, cyano, nitro, carboxy, carboalkoxy of 2-7 carbon atoms, carboalkyl of 2-7 carbon atoms, phenoxy, phenyl, thiophenoxy, benzoyl, benzyl, amino, alkylamino of 1-6 carbon atoms, dialkylamino of 2 to 12 carbon atoms, phenylamino, benzylamino, alkanoylamino of 1-6 carbon atoms, alkenoylamino of 3-8 carbon atoms, alkynoylamino of 3-8 carbon atoms, carboxyalkyl of 2-7 carbon atoms, carboalkoxyalkyl of 3-8 carbon atoms, aminoalkyl of 1-5 carbon atoms, N-alkylaminoalkyl of 2-9 carbon atoms, N,N-dialkylaminoalkyl of 3-10 carbon atoms, N-alkylaminoalkoxy of 2-9 carbon atoms, N,N-dialkylaminoalkoxy of 3-10 carbon atoms, mercapto, and benzoylamino; or
L is a 5- or 6-membered heteroaryl ring where the heteroaryl ring contains 1 to 3 heteroatoms selected from N, O, and S, with the proviso that the heteroaryl ring does not contain O—O, S—S, or S—O bonds, and where the heteroaryl ring is optionally mono- or di-substituted with a substituent selected from the group consisting of halogen, oxo, thio, alkyl of 1-6 carbon atoms, alkenyl of 2-6 carbon atoms, alkynyl of 2-6 carbon atoms, azido, hydroxyalkyl of 1-6 carbon atoms, halomethyl, alkoxymethyl of 2-7 carbon atoms, alkanoyloxymethyl of 2-7 carbon atoms, alkoxy of 1-6 carbon atoms, alkylthio of 1-6 carbon atoms, hydroxy, trifluoromethyl, cyano, nitro, carboxy, carboalkoxy of 2-7 carbon atoms, carboalkyl of 2-7 carbon atoms, phenoxy, phenyl, thiophenoxy, benzoyl, benzyl, amino, alkylamino of 1-6 carbon atoms, dialkylamino of 2 to 12 carbon atoms, phenylamino, benzylamino, alkanoylamino of 1-6 carbon atoms, alkenoylamino of 3-8 carbon atoms, alkynoylamino of 3-8 carbon atoms, carboxyalkyl of 2-7 carbon atoms, carboalkoxyalkyl of 3-8 carbon atoms, aminoalkyl of 1-5 carbon atoms, N-alkylaminoalkyl of 2-9 carbon atoms, N,N-dialkylaminoalkyl of 3-10 carbon atoms, N-alkylaminoalkoxy of 2-9 carbon atoms, N,N-dialkylaminoalkoxy of 3-10 carbon atoms, mercapto, and benzoylamino;
V is ethylene or acetylene;
R₄ is alkyl of 1 to 6 carbons;
Z is NR^{z′}, O or S, wherein R^{z′} is H or C₁-C₆ alkyl;
R₁, G₂, and R₃ are each, independently, hydrogen, halogen, alkyl of 1-6 carbon atoms, alkenyl of 2-6 carbon atoms, alkynyl of 2-6 carbon atoms, alkenyloxy of 2-6 carbon atoms, alkynyloxy of 2-6 carbon atoms, hydroxymethyl, halomethyl, alkanoyloxy of 1-6 carbon atoms, alkenoyloxy of 3-8 carbon atoms, alkynoyloxy of 3-8 carbon atoms, alkanoyloxymethyl of 2-7 carbon atoms, alkenoyloxymethyl of 4-9 carbon atoms, alkynoyloxymethyl of 4-9 carbon atoms, alkoxymethyl of 2-7 carbon atoms, alkoxy of 1-6 carbon atoms, alkylthio of 1-6 carbon atoms, alkylsulphinyl of 1-6 carbon atoms, alkylsulphonyl of 1-6 carbon atoms, alkylsulfonamido of 1-6 carbon atoms, alkenylsulfonamido of 2-6 carbon atoms, alkynylsulfonamido of 2-6 carbon atoms, hydroxy, trifluoromethyl, trifluoromethoxy, cyano, nitro, carboxy, carboalkoxy of 2-7 carbon atoms, carboalkyl of 2-7 carbon atoms, phenoxy, phthalimide, phenyl, thiophenoxy, benzyl, amino, hydroxyamino, alkoxyamino of 14 carbon atoms, alkylamino of 1-6 carbon atoms, dialkylamino of 2 to 12 carbon atoms, N-alkylcarbamoyl, N,N-dialkylcarbamoyl, N-alkyl-N-alkenylamino of 4 to 12 carbon atoms, N,N-dialkenylamino of 6-12 carbon atoms, phenylamino, benzylamino, 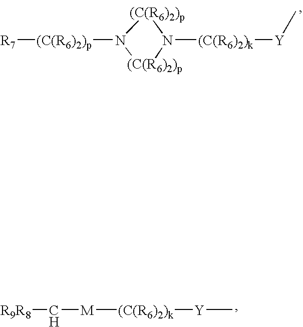
R₇—(C(R₆)₂)_{g}—Y—, R₇—(C(R₆)₂)ₚ-M-(C(R₆)₂)ₖ—Y—, or Het-(C(R₆)₂)_{q}W—(C(R₆)₂—Y—;
or R₁ and R₃ are as defined above and G₂ is R₂—NH—;
or if any of the substituents R₁, G₂ or R₃ are located on contiguous carbon atoms then they may be taken together as the divalent radical —O—C(R₆)₂—O—;
Y is a divalent radical selected from the group consisting of 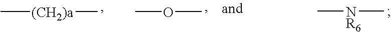
R₇ is —NR₆R₆, —OR₆, -J, —N(R₆)₃⁺, or —NR₆(OR₆);
M is >NR₆, —O—, >N—(C(R₆)₂)ₚNR₆R₆, or >N—(C(R₆)₂)ₚ—OR₆;
W is >NR₆, —O— or is a bond;
Het is is selected from the group consisting of morpholine, thiomorpholine, thiomorpholine S-oxide, thiomorpholine S,S-dioxide, piperidine, pyrrolidine, aziridine, pyridine, imidazole, 1,2,3-triazole, 1,2,4-triazole, thiazole, thiazolidine, tetrazole, piperazine, furan, thiophene, tetrahydrothiophene, tetrahydrofuran, dioxane, 1,3-dioxolane, tetrahydropyran, and 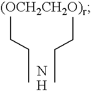
wherein Het is optionally mono- or di-substituted on carbon or nitrogen with R₆, optionally mono- or di-substituted on carbon with hydroxy, —N(R₆)₂, or —OR₆, optionally mono or di-substituted on carbon with the mono-valent radicals —(C(R₆)₂)ₛOR₆ or —(C(R₆)₂)ₛN(R₆)₂, and optionally mono or di-substituted on a saturated carbon with divalent radicals —O— or —O(C(R₆)₂)ₛO—;
R₆ is hydrogen, alkyl of 1-6 carbon atoms, alkenyl of 2-6 carbon atoms, alkynyl of 2-6 carbon atoms, cycloalkyl of 1-6 carbon atoms, carboalkyl of 2-7 carbon atoms, carboxyalkyl (2-7 carbon atoms), phenyl, or phenyl optionally substituted with one or more halogen, alkoxy of 1-6 carbon atoms, trifluoromethyl, amino, alkylamino of 1-3 carbon atoms, dialkylamino of 2-6 carbon atoms, nitro, cyano, azido, halomethyl, alkoxymethyl of 2-7 carbon atoms, alkanoyloxymethyl of 2-7 carbon atoms, alkylthio of 1-6 carbon atoms, hydroxy, carboxyl, carboalkoxy of 2-7 carbon atoms, phenoxy, phenyl, thiophenoxy, benzoyl, benzyl, phenylamino, benzylamino, alkanoylamino of 1-6 carbon atoms, or alkyl of 1-6 carbon atoms; with the proviso that the alkenyl or alkynyl moiety is bound to a nitrogen or oxygen atom through a saturated carbon atom;
R₂, is selected from the group consisting of 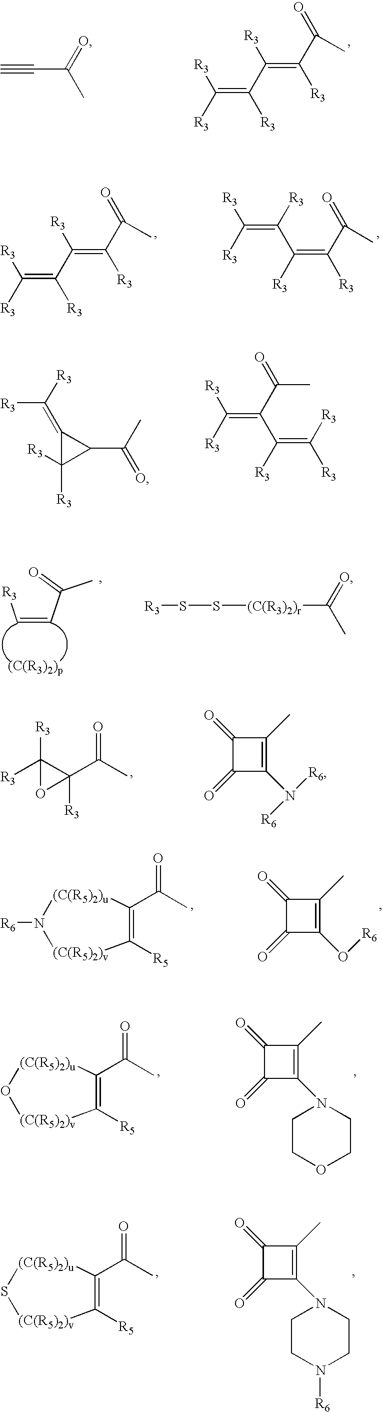 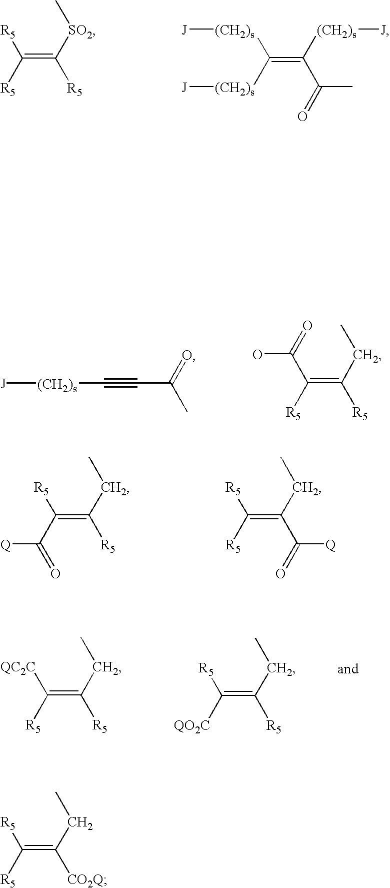
R₃ is independently hydrogen, alkyl of 1-6 carbon atoms, carboxy, carboalkoxy of 1-6 carbon atoms, phenyl, carboalkyl of 2-7 carbon atoms, 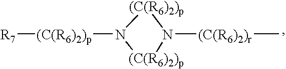
R₇—(C(R₆)₂)ₛ—,
R₇—(C(R₆)₂)ₚ-M-(C(R₆)₂)ᵣ—, R₈R₉—CH—
M-(C(R₆)₂)ᵣ—, or
Het-(C(R₆)₂)_{q}—W—(C(R₆)₂)ᵣ—;
R₅ is independently hydrogen, alkyl of 1-6 carbon atoms, carboxy, carboalkoxy of 1-6 carbon atoms, phenyl carboalkyl of 2-7 carbon atoms, 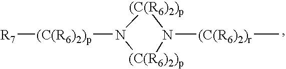
R₇—(C(R₆)₂)ₛ—,
R₇—(C(R₆)₂)ₚ-M-(C(R₆)₂)ᵣ—, R₈R₉—CH—
M-(C(R₆)₂)ᵣ—, or
Het-(C(R₆)₂)_{q}—W—C(R₆)₂)ᵣ—;
R₈, and R₉ are each independently —(C(R₆)₂)ᵣNR₆R₆, or —(C(R₆)₂)ᵣOR₆;
J is independently hydrogen, chlorine, fluorine, or bromine;
Q is aklyl of 1-6 carbon atoms or hydrogen;
a=0 or 1;
g=1-6;
k=0-4;
n is 0-1;
m is 0-3;
p=2-4;
q=0-4;
r=1-4;
s=1-6;
u=0-4 and v=0-4, wherein the sum of u+v is 2-4;
x=0-3;
y=0-1; and
z=0-3;
or a pharmaceutically acceptable salt thereof.

15. The method of claim 14, further comprising the following provisos:
when R₆ is alkenyl of 2-7 carbon atoms or alkynyl of 2-7 carbon atoms, such alkenyl or alkynyl moiety is bound to a nitrogen or oxygen atom through a saturated carbon atom;
when Y is —NR₆— and R₇ is —NR₆R₆, —N(R₆)₃.N(R₈)₃⁺, or —NR₆(OR₆), then g=2-6;
when M is —O— and R₇ is —OR₆ then p=1-4;
when Y is —NR₆— then k=2-4;
when Y is —O— and M or W is —O— then k=1-4;
when W is not a bond with Het bonded through a nitrogen atom then q=2-4;
when W is a bond with Het bonded through a nitrogen atom and Y is —O— or —NR₆— then k=2-4; and
provided that L can be an unsubstituted phenyl ring only when m>0 and T is not —CH₂NH— or —CH₂O—.
